(19) United States
(12) Patent Application Publication
(10) Pub. No.: US 2025/0360057 A1
(43) Pub. Date: Nov. 27, 2025

(54) ENHANCING DENTIN BONDING DURABILITY USING QUATERNARY PYRIDINIUM SALTS

(71) Applicant: ADA Science and Research Institute LLC, Chicago, IL (US)

(72) Inventors: Xiaohong WANG, North Potomac, MD (US); Nicholas L. Hammond, Baltimore, MD (US)

(73) Assignee: ADA Science and Research Institute LLC, Chicago, IL (US)

(21) Appl. No.: 18/872,720

(22) PCT Filed: Jun. 7, 2023
(86) PCT No.: PCT/US2023/024709

§ 371 Date: Dec. 6, 2024

Related U.S. Application Data
(60) Provisional application No. 63/350,288, filed on Jun. 8, 2022.

(51) Int. Cl. A61K 6/30; A61K 6/60; C08L 33/10

(52) U.S. Cl. CPC A61K 6/30; A61K 6/60; C08L 33/10

(57) ABSTRACT

Disclosed are quaternary pyridinium salt additives for dental resins and adhesives that enhance their stability and their dentin bonding durability. At additive levels between about 0.1 and 1.25 wt %, these additives have been found to increase at least one of the following: (i) the stability of dental resins and adhesives against enzymatic challenge, (ii) the bonding durability with dentin, and/or (iii) the degree of conversion.

### CROSS-REFERENCE TO RELATED APPLICATION

This application claims priority to U.S. Provisional Application No. 63/350,288, filed Jun. 8, 2022, the entire contents of which is hereby incorporated by reference in its entirety.

### BACKGROUND

Adhesive dentistry is based on the development of materials which establish an effective bond with the tooth tissues. Successful adhesive bonding depends on the chemistry of the adhesive, on appropriate clinical handling of the material as well as on the knowledge of the morphological changes caused on dental tissue by different bonding procedures. One of the major reasons for short service life of dental restoratives is the bonding failure within the adhesive layer.

There is therefore a need in providing improved adhesives and resins to improve the service life of dental restoratives.

### BRIEF SUMMARY OF THE INVENTION

In various aspects, a dental composition is provided. The dental composition includes a dental adhesive or resin, and at least one compound of formula I,

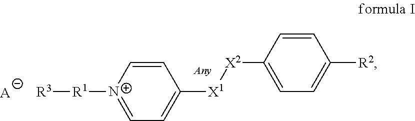

wherein:
A⁻ is an anion;
X¹ and X² are each independently selected from the group consisting of O, N, C, CH, and CH₂;
[symbol] represents a single, double, or triple bond;
R¹ is selected from the group consisting of —CₙH₍₂ₙ₊₁₎ and —CₙH₍₂ₙ₋₁₎;
R² is selected from the group consisting of —OC₂₋₈ alkenyl, methacrylate, acrylate, styrene, vinyl benzyl, isobutyrate, and —OH;
n is 2, 3, 4, 5, 6, 7, 8, 9, 10, 11, 12, 13, 14, 15, 16, 17, 18, 19, or 20;
wherein the composition optionally comprises at least one (third moiety) R³ attached to R¹, wherein R³ is independently selected from the group consisting of methacrylate, acrylate, styrene, vinyl benzyl, F, Cl, Br, and I; and
wherein the compound of formula I is present in an amount of 0.1 to 1.25% (w/w) relative to the weight of the dental composition.

In certain embodiments, a method of using a dental composition is also provided. The method includes:
applying a dental composition to an oral surface to form a coated oral surface, wherein the dental composition contains a dental adhesive or resin and at least one compound of formula I,

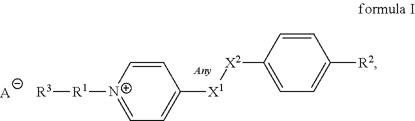

wherein:
   A⁻ is an anion;
   X¹ and X² are each independently selected from the group consisting of O, N, C, CH, and CH₂;
   [symbol] represents a single, double, or triple bond;
   R¹ is selected from the group consisting of —CₙH₍₂ₙ₊₁₎ and —CₙH₍₂ₙ₋₁₎;
   R² is selected from the group consisting of —OC₂₋₈ alkenyl, methacrylate, acrylate, styrene, vinyl benzyl, isobutyrate, and —OH;
   n is 2,3,4,5, 6, 7, 8, 9, 10, 11, 12, 13, 14, 15, 16, 17, 18, 19, or 20;
   wherein the composition optionally comprises at least one (third moiety) R³ attached to R¹, wherein R³ is selected from the group consisting of methacrylate, acrylate, styrene, vinyl benzyl, F, Cl, Br, and I;
   wherein the compound of formula I is present in an amount of 0.1 to 1.25% (w/w) relative to the weight of the dental composition;
and
curing the coated oral surface.

Surprisingly and advantageously, the dental compositions described herein can increase at least one of the following: (i) stability of dental resins and adhesives against enzymatic challenge, (ii) bonding durability with dentin, and/or (iii) degree of conversion.

### BRIEF DESCRIPTION OF THE FIGURES

The drawings illustrate generally, by way of example, but not by way of limitation, various embodiments of the present application.

FIG. 1A illustrates an illustrative synthesis of a compound of formula I, Azo-QPS-R, where R is R³—R¹— as defined herein, according to some embodiments.

FIG. 1B illustrates an illustrative synthesis of a compound of formula I, according to some embodiments.

FIG. 1C shows an illustrative compound of formula I, according to some embodiments.

FIG. 1D shows an illustrative compound of formula I, according to some embodiments

FIG. 1E shows an illustrative compound of formula I, according to some embodiments

FIG. 1F shows an illustrative compound of formula I, according to some embodiments.

FIG. 2 is a graph showing the effect on degree of vinyl conversion for two adhesive materials incorporating an additive according to the present invention (Azo-QPS-C16).

FIG. 3 is a graph showing the effect of an additive of the present invention on the glass transition temperature of dental materials.

FIG. 4 is a graph showing stability measurements as a function of time, comparing an adhesive with no additive with the same adhesive having an additive according to the present invention (Scotchbond—SB/Azo-QPS-C16).

FIG. 5 is a graph showing stability measurements as a function of time, comparing an adhesive with no additive with the same adhesive having an additive according to the present invention (B6H4/Azo-QPS-C16).

FIG. 6 is a graph showing the effect of two different additives on the stability measurements of an adhesive as a function of time (B6H4, MA-C6-Azo-Py-iBu & MA-CB13-Azo-QPS-iBu).

FIG. 7 is a graph showing the effect of two different additives on the stability measurements of another adhesive as a function of time (SB, MA-C6-Azo-Py-iBu & MA-CB13-Azo-QPS-iBu).

FIG. 8 is a graph showing the effect of an additive on the stability of PBNT adhesives as a function of time (additive is Azo-QPS-C16).

FIG. 9 is a graph showing the effect of an additive on the stability of SBP adhesives as a function of time (additive is Azo-QPS-C16).

FIG. 10 is a graph showing the effect of an additive on the stability of SUP adhesives as a function of time (additive is Azo-QPS-C16).

FIG. 11 is a graph showing the effect of an additive on the stability of EBSE adhesives as a function of time (additive is Azo-QPS-C16).

FIG. 12 is a graph showing the effect of an additive on the stability of various other adhesives as a function of time (additive is Azo-QPS-C16).

FIG. 13 is a graph showing the measured shear bond strength for adhesives with varying quantities of additive.

FIG. 14 is a graph showing micro-tensile bond strength (pTBS) of adhesives with and without additives.

FIG. 15 shows a method of determining the biostability of cured resins containing a dental adhesive and/or resin and a compound of formula I.

FIGS. 16A-16D shows results for an example of a resin surface prepared using the method shown in FIG. 15. FIG. 16A is an optical microscope image showing a test surface prepared according to the method shown in FIG. 15 with an array of metal-covered regions (dark grey) and bare-resin channels (light grey). FIG. 16B shows an atomic force microscopy image for the exemplary surface shown in FIG. 16A. The inset indicates the height. FIG. 16C shows an AFM image for the exemplary surface shown in FIG. 16A after 72 h incubation in pseudochlorine esterase, indicating a cross-section (dashed white line) across which a depth profile was measured. FIG. 16D shows a height-distance depth profile for the cross section identified in FIG. 16 C of the exemplary surface after 72 h incubation in pseudochlorine. FIGS. 16A-16D taken from Wang X, Song S, Chen L, Stafford C M, Sun P. Acta Biomater., 2018, 74, 326.

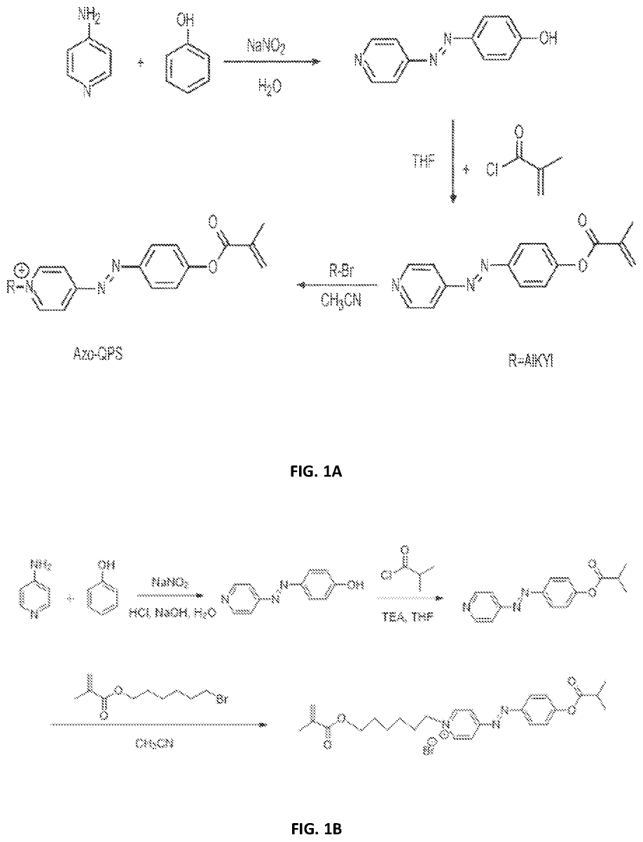

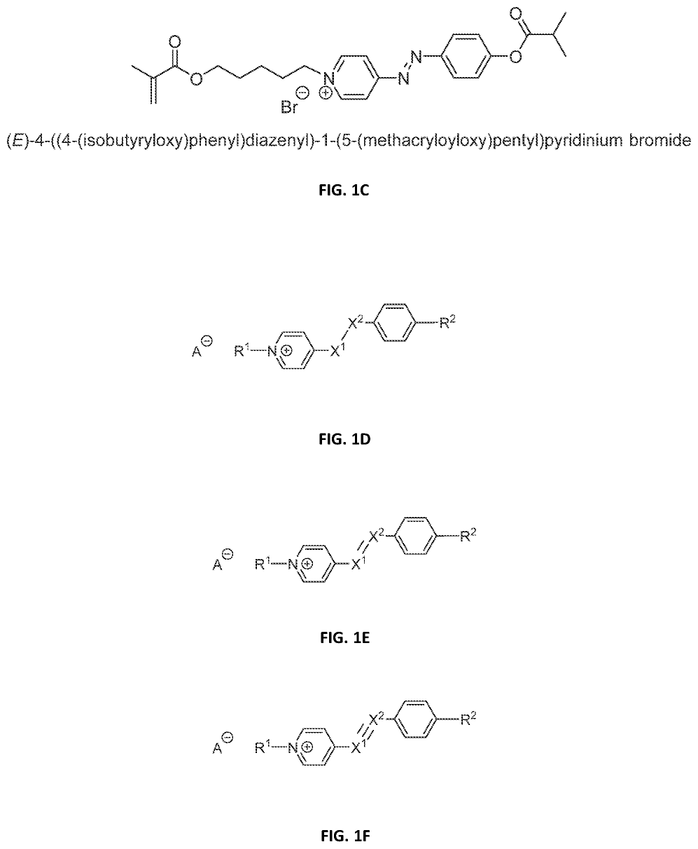

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include notjust about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The substitution can be direct substitution, whereby the hydrogen atom is replaced by a functional group or substituent, or an indirect substitution, whereby an intervening linker group replaces the hydrogen atom, and the substituent or functional group is bonded to the intervening linker group. A non-limiting example of direct substitution is: RR-H→RR-Cl, wherein RR is an organic moiety/fragment/molecule. A non-limiting example of indirect substitution is: RR-H→RR-(LL)_{zz}-Cl, wherein RR is an organic moiety/fragment/molecule, LL is an intervening linker group, and ‘zz’ is an integer from 0 to 100 inclusive. When zz is 0, LL is absent, and direct substitution results. The intervening linker group LL is at each occurrence independently selected from the group consisting of —H, —O—, —OR, —S—, —S(═O)—, —S(═O)₂—, —SR, —N(R)—, —NR₂, —CR═, —C═, —CH₂—, —CHR—, —CR₂—, —CH₃, —C(═O)—, —C(═NR)—, and combinations thereof. (LL)_{zz} can be linear, branched, cyclic, acyclic, and combinations thereof.

The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl.

Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenvlacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbomyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbomyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “heterocycloalkyl” as used herein refers to a cycloalkyl group as defined herein in which one or more carbon atoms in the ring are replaced by a heteroatom such as O, N, S, P, and the like, each of which may be substituted as described herein if an open valence is present, and each may be in any suitable stable oxidation state.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. The term heterocyclyl includes rings where a CH₂ group in the ring is replaced by one or more C═O groups, such as found in cyclic ketones, lactones, and lactams. Examples of heterocyclyl groups containing a C═O group include, but are not limited to, β-propiolactam, γ-butyrolactam, δ-valerolactam, and ε-caprolactam, as well as the corresponding lactones. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. A heterocyclyl ring designated C_{x-y} can be any ring containing ‘x’ members up to ‘y’ members, including all intermediate integers between ‘x’ and ‘y’ and that contains one or more heteroatoms, as defined herein. In a ring designated C_{x-y}, all non-heteroatom members are carbon. Heterocyclyl rings designated C_{x-y} can also be polycyclic ring systems, such as bicyclic or tricyclic ring systems. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanvl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-vl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about I to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g, the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonvl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxvcyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

As used herein, the term “C₆₋₁₀-5-6 membered heterobiaryl” means a C₆₋₁₀ aryl moiety covalently bonded through a single bond to a 5- or 6-membered heteroaryl moiety. The C₆₋₁₀ aryl moiety and the 5-6-membered heteroaryl moiety can be any of the suitable aryl and heteroaryl groups described herein. Non-limiting examples of a C₆₋₁₀-5-6 membered heterobiaryl include

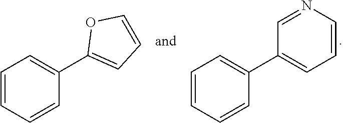 [SMILES: c1ccc(-c2ccco2)cc1 ; c1ccc(-c2cccnc2)cc1]

When the C₆₋₁₀-5-6 membered heterobiaryl is listed as a substituent (e.g., as an “R” group), the C₆₋₁₀-5-6 membered heterobiaryl is bonded to the rest of the molecule through the C₆₋₁₀ moiety.

As used herein, the term “5-6 membered-C₆₋₁₀ heterobiaryl” is the same as a C₆₋₁₀-5-6 membered heterobiaryl, except that when the 5-6 membered-C₆₋₁₀ heterobiaryl is listed as a substituent (e.g., as an “R” group), the 5-6 membered-C₆₋₁₀ heterobiaryl is bonded to the rest of the molecule through the 5-6-membered heteroaryl moiety.

As used herein, the term “C₆₋₁₀—C₆₋₁₀ biaryl” means a C₆₋₁₀ aryl moiety covalently bonded through a single bond to another C₆₋₁₀ aryl moiety. The C₆₋₁₀ aryl moiety can be any of the suitable aryl groups described herein. Non-limiting example of a C₆₋₁₀—C₆₋₁₀ biaryl include biphenyl and binaphthyl.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise.

Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

As used herein, an “oral surface” refers to a soft or hard surface in the oral environment. Hard surfaces typically include tooth structure including, for example, natural and artificial tooth surfaces, bone, and the like.

As used herein, “dental composition” refers to an unfilled material (i.e., total dental composition except for filler) or filled material (e.g., a dental cement or restoration) capable of adhering or being bonded to an oral surface.

As used herein, “orthodontic appliance” refers to any device intended to be bonded to a tooth structure, including, but not limited to, orthodontic brackets, buccal tubes, lingual retainers, orthodontic bands, bite openers, buttons, and cleats. The appliance has a base for receiving adhesive and it can be a flange made of metal, plastic, ceramic, or combinations thereof. Alternatively, the base can be a custom base formed from cured adhesive layer(s) (i.e. single or multi-layer adhesives).

As used herein, “dental article” refers to an article that can be adhered (e.g., bonded) to a tooth structure. Dental articles include, for example, crowns, bridges, veneers, inlays, onlays, fillings, orthodontic appliances and devices.

As used herein, “additive” refers to one or more compounds of formula I.

As used herein, “dental adhesive and/or resin” refers to a substance capable of safely and securely adhering to an oral surface, resin composites, orthodontic appliance, and/or a dental article. The dental adhesive and/or resin is capable of polymerizing under thermal or photochemical conditions to cure and harden.

As used herein, “degree of conversion” or “DC” refers to the degree of vinyl conversion (DVC).

### Preparation of Compounds of Formula I

Compounds of formula I or otherwise described herein can be prepared by the general schemes described herein, using the synthetic method known by those skilled in the art. The following examples illustrate non-limiting embodiments of the compound(s) described herein and their preparation.

In various embodiments, a dental composition is provided. The dental composition includes a dental adhesive or resin and at least one compound of formula I,

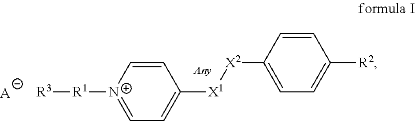

wherein:
A⁻ is an anion;
X¹ and X² are each independently selected from the group consisting of O, N, C, CH, and CH₂;
[symbol] represents a single, double, or triple bond;
R¹ is selected from the group consisting of —CₙH₍₂ₙ₊₁₎ and —CₙH₍₂ₙ₋₁₎;
R² is selected from the group consisting of —OC₂₋₈ alkenyl, methacrylate, acrylate, styrene, vinyl benzyl, isobutyrate, and —OH;
n is 2, 3, 4, 5, 6, 7, 8, 9, 10, 11, 12, 13, 14, 15, 16, 17, 18, 19, or 20;
wherein the composition optionally comprises at least one R³ attached to R¹, wherein R³ is selected from the group consisting of methacrylate, acrylate, styrene, vinyl benzyl, F, Cl, Br, and I;
wherein the compound of formula I is present in an amount of 0.1 to 1.25% (w/w) relative to the weight of the dental composition.

When R¹ is CₙH₍₂ₙ₊₁₎, variable R¹ can be straight or branched alkyl. When R¹ is CₙH₍₂ₙ₋₁₎, variable Ri can be straight or branched alkenyl. The methacrylate, acrylate, styrene, vinyl benzyl, isobutyrate groups can be attached in the following ways in the compound of formula I:

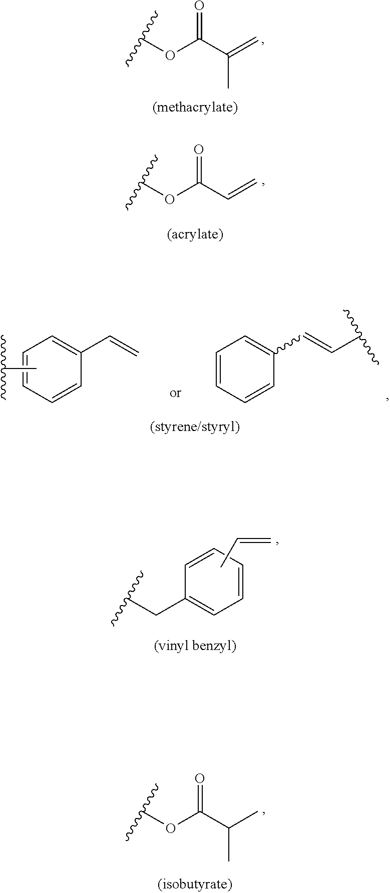

wherein the wavy bond indicates either cis or trans isomerism, and the wavy line

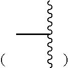

indicates the point of attachment. In various embodiments, the compound of formula I contains at least one carbon-carbon double bond capable of polymerizing with the dental adhesive and/or resin, such as a dental adhesive and/or resin that is ethylenically unsaturated. The R³ group, when present, is connected to R¹ through any open valence, such as replacing any C—H bond in R¹ with a C—R³ bond. In various embodiments, R³ is connected to the terminal carbon in R¹. In various embodiments, R³ is absent.

In various embodiments, [symbol] represents a double bond. In various embodiments, [symbol] represents a single bond. In various embodiments, [symbol] represents a triple bond.

In various embodiments, X¹ and X² are not both O (oxygen).

In various embodiments, if one of X¹ and X² is N (nitrogen), then the other is nitrogen or carbon (such as CH).

In various embodiments, if one of X¹ and X² is N (nitrogen), then [symbol] represents a double bond.

In various embodiments, if one of X¹ and X² is O, then [symbol] represents a single bond.

In various embodiments, X^{t} is N and X² is N, and [symbol] represents a double bond.

In various embodiments, if both X¹ and X² are C, then [symbol] represents a triple bond.

In various embodiments, if both X¹ and X² are CH, then [symbol] represents a double bond.

In various embodiments, if both X¹ and X² are CH₂, then [symbol] represents a single bond.

In various embodiments, A⁻ is selected from the group consisting of NO₃⁻, NO₂⁻, SCN⁻, CN⁻, PO₄³⁻, SO₄²⁻, F⁻, Cl⁻, Br⁻, I⁻, BF₄⁻, AsF₆⁻, SbF₆⁻, CH₃CO₂⁻, CF₃SO₃⁻, (CF₃SO₂)₂N⁻, (CF₃SO₂)₃C⁻, and CF₃COO⁻. In various embodiments, A⁻ is Br⁻. Additionally, the anion can be the conjugate base of any pharmaceutically acceptable acid addition salt described herein. In various embodiments, n is 2, 3, 4, 5, 6, 7, 8, 9, 10, 11, 12, 13, 14, 15, 16, 17, 18, 19, or 20.

In various embodiments, the compound of formula I is present in an amount of 0.1 to 1.25% (w/w) relative to the weight of the dental composition. In various embodiments, the compound of formula I is present in an amount of 0.2 to 1% (w/w) relative to the weight of the dental composition. In various embodiments, the compound of formula I is present in an amount of 0.25 to 0.5% (w/w) relative to the weight of the dental composition. In some embodiments, the compound of formula I is present in an amount of at least, equal to, or greater than about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, or about 1.25% (w/w) relative to the weight of the dental composition.

In various embodiments, the compound of formula I is selected from the group consisting of

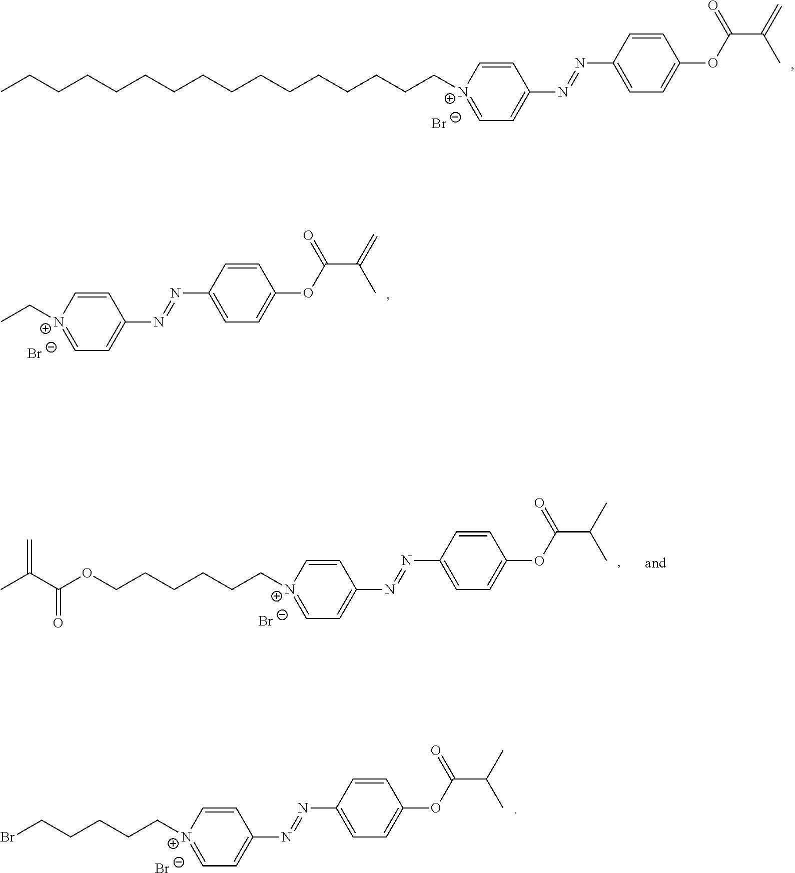

In various embodiments, a cured composition is provided. The cured composition includes a polymerized product of a dental adhesive or resin and at least one compound of formula I,

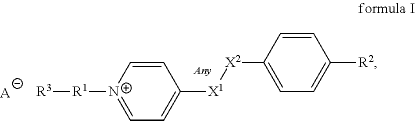

wherein:
A⁻ is an anion;
X¹ and X² are each independently selected from the group consisting of O, N, C, CH, and CH₂;
[symbol] represents a single, double, or triple bond;
R¹ is selected from the group consisting of —CₙH₍₂ₙ₊₁₎ and —CₙH₍₂ₙ₋₁₎;
R² is selected from the group consisting of —OC₂₋₈ alkenyl, methacrylate, acrylate, styrene, vinyl benzyl, isobutyrate, and —OH;
n is 2, 3, 4, 5, 6, 7, 8, 9, 10, 11, 12, 13, 14, 15, 16, 17, 18, 19, or 20;
wherein the composition optionally comprises at least one third moiety R³ attached to R¹, wherein R³ is selected from the group consisting of methacrylate, acrylate, styrene, vinyl benzyl, F, Cl, Br, and I;
wherein the compound of formula I is present in an amount of 0.1 to 1.25% (w/w) relative to the weight of the dental composition.

In various embodiments, in the polymerized product of a dental adhesive and/or resin and a compound of formula I, the compound of formula I is selected from the group consisting of:

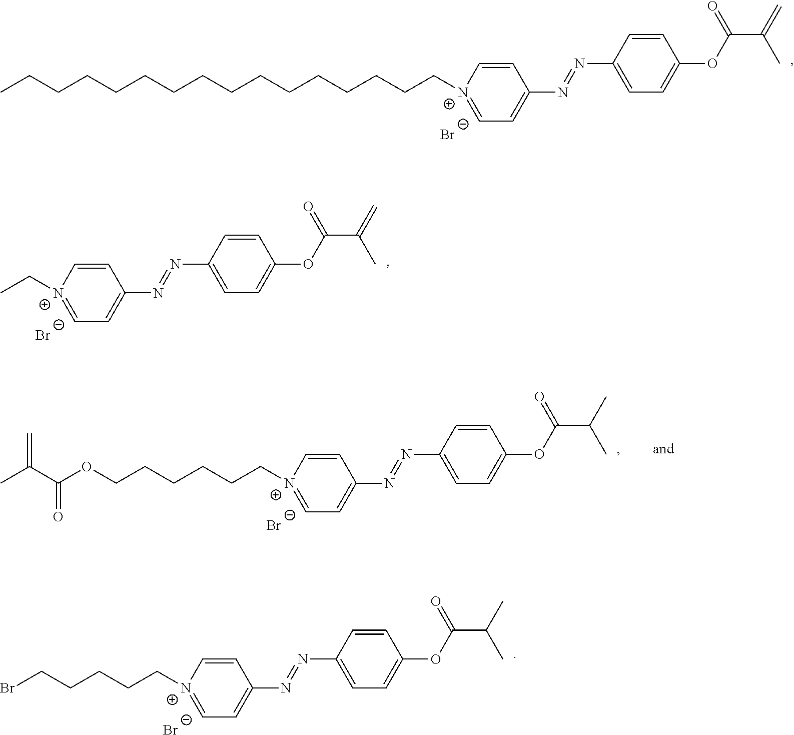

The polymerized product, which can also be referred to as a cured product, can be formed by thermally or photochemically polymerizing the dental composition described herein. In various embodiments, the polymerized product has a higher degree of vinyl conversion than if the compound of formula I was absent. In some embodiments, the increase in vinyl conversion (DC) is at least about or equal to 1, 1.5, 2, 2.5, 3, 3.5, 4, 4.5, 5, 5.5, 6, 6.5, 7, 7.5, 8, 8.5, 9, 9.5, or 10% compared to the vinyl conversion in a dental composition lacking a compound of formula I.

Surprisingly and unexpectedly, compounds of formula I, when incorporated into dental adhesives and/or resins results in at least one of the following: improved durability of adhesives (from thermal cycling tests and micro-tensile bond strength measurements), improved stability against enzymatic attack, improved stability against bacterial attack, increase in the degree of vinyl conversion, and/or narrower peaks in the glass transition temperature (indicating more cross-linking, which improves stability).

Unexpectedly, the weight % incorporation of the compound of formula I into the resin or adhesive drives these properties. In various embodiments, when compounds of formula I are incorporated at levels greater than about 1.25 wt %, the polymerization starts to reduce due to lack of homopolymerization with the presence of an additive, which decreases the degree of conversion (DC). In various embodiments, when compounds of formula I are present in a dental adhesive and/or resin composition at greater than or equal to about 2, 3, 4, 5, 6, 7, 8, 9, or 10% wt % relative to the weight of the dental composition, the superior properties of the dental compositions described herein are not observed. The superior properties include, but are not limited to, improved durability of adhesives, improved stability against enzymatic and/or bacterial attack, improved stability against bacterial attack increase in the degree of vinyl conversion, and/or narrower peaks in the glass transition temperature.

In various embodiments, the polymerized product has a higher biostability than if the compound of formula I is absent from the dental composition. Biostability can be measured by determining the changes in the amount of cured dental composition that is degraded in the presence of an enzyme such as pseudocholine esterase from equine serum (PCE). Without being bound by theory, higher amounts of unpolymerized vinyl groups leads to greater potential for resin degradation. Specifically, the lower DC resins contain more unconverted (uncured) dental adhesive and/or resin, which exist in the resin as leachable monomers with two vinyl groups or pendants to the resin network. These pendants may exist as monomers with one unpolymerized double bond, oligomers or resin blocks that are dangling on the main cross-linked resin network. In comparison to segments in the main cross-linked resin network, these pendants can be more readily cut away from the resin network because less ester function groups need to be hydrolyzed and fewer crosslinking points to be broken.

In various embodiments, polymerized product has a higher durability than if the compound of formula I was absent. Durability of the adhesive bonding to dentin can be assessed, in a non-limiting example, as described herein by thermal cycling of the materials, followed by measurement of the microtensile bond strength (pTBS).

In various embodiments, compounds described in U.S. Pat. Nos. 10,836,726 and 11,104,647, which are both incorporated by reference herein in their entireties, can be used as additives as described herein.

The dental adhesives and/or resins that are combined with compounds of formula I can include a wide variety of ethylenically unsaturated compounds (with or without acid functionality), epoxy-functional (meth)acrylate resins, vinyl ethers, glass ionomer cements, and the like.

The dental adhesives and/or resins can include compounds having free radically reactive functional groups that may include monomers, oligomers, and polymers having one or more ethylenically unsaturated group. Suitable compounds contain at least one ethylenically unsaturated bond and are capable of undergoing addition polymerization. Examples of useful ethylenically unsaturated compounds include acrylic acid esters, methacrylic acid esters, hydroxy-functional acrylic acid esters, hydroxy-functional methacrylic acid esters, and combinations thereof. Such free radically polymerizable compounds include mono-, di- or poly-(meth)acrylates (i.e., acrylates and methacrylates) such as, methyl (meth)acrylate, ethyl (meth)acrylate, isopropyl (meth)acrylate, n-hexyl (meth)acrylate, stearyl (meth)acrylate, allyl (meth)acrylate, glycerol tri(meth)acrylate, ethyleneglycol di(meth)acrylate, diethyleneglycol di(meth)acrylate, triethyleneglycol di(meth)acrylate, 1,3-propanediol di(meth)acrylate, trimethylolpropane tri(meth)acrylate, 1,2,4-butanetriol tri(meth)acrylate, 1,4-cyclohexanediol di(meth)acrylate, pentaerythritol tetra(meth)acrylate, sorbitol hex(meth)acrylate, tetrahydrofurfuryl (meth)acrylate, bis[l-(2-acryloxy)]-p-ethoxyphenyldimethylmethane, bis[1-(3-acryloxy-2-hydroxy)]-p-propoxyphenyldimethylmethane, ethoxylated bisphenolA di(meth)acrylate, and trishydroxyethyl-isocyanurate tri(meth)acrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500); and vinyl compounds such as styrene, diallyl phthalate, divinyl succinate, divinyl adipate and divinyl phthalate. Other suitable free radically polymerizable compounds include siloxane-functional (meth)acrylates. Mixtures of two or more free radically polymerizable compounds can be used if desired.

The dental adhesive and/or resin can also contain hydroxyl groups and ethylenically unsaturated groups in a single molecule. Examples of such materials include hydroxyalkyl (meth)acrylates, such as 2-hydroxyethyl (meth)acrylate and 2-hydroxypropyl (meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-ethacryloxypropoxy)phenyl]propane (bisGMA). Suitable ethylenically unsaturated compounds are also available from a wide variety of commercial sources, such as Sigma-Aldrich, St. Louis.

In certain embodiments dental adhesive and/or resin can include bisGMA, UDMA (urethane dimethacrylate), TEGDMA (triethyleneglycol dimethacrylate), and combination thereof.

The compositions described herein can include one or more curable components in the form of ethylenically unsaturated compounds with acid functionality. Such components contain acidic groups and ethylenically unsaturated groups in a single molecule. When present, the polymerizable component optionally comprises an ethylenically unsaturated compound with acid functionality. In some embodiments, the acid functionality includes an oxyacid (i.e., an oxygen-containing acid) of carbon, sulfur, phosphorous, or boron.

As used herein, ethylenically unsaturated compounds with acid functionality is meant to include monomers, oligomers, and polymers having ethylenic unsaturation and acid and/or acid-precursor functionality. Acid-precursor functionalities include, for example, anhydrides, acid halides, and pyrophosphates. The acid functionality can include carboxylic acid functionality, phosphoric acid functionality, phosphonic acid functionality, sulfonic acid functionality, or combinations thereof.

Ethylenically unsaturated compounds with acid functionality include, for example, α,β-unsaturated acidic compounds such as glycerol phosphate mono(meth)acrylates, glycerol phosphate di(meth)acrylates, hydroxyethyl (meth)acrylate (e.g., HEMA) phosphates, bis((meth)acryloxyethyl)phosphate, ((meth)acryloxypropyl)phosphate, bis((meth)acryloxypropyl)phosphate, bis((meth)acryloxy)propyloxy phosphate, (meth)acryloxyhexyl phosphate, bis((meth)acryloxyhexyl)phosphate, (meth)acryloxyoctyl phosphate, bis((meth)acryloxyoctyl)phosphate, (meth)acryloxydecyl phosphate, bis((meth)acryloxydecyl)phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, may be used as components. Also monomers, oligomers, and polymers of unsaturated carbonic acids such as (meth)acrylic acids, aromatic (meth)acrylated acids (e.g., methacrylated trimellitic acids), and anhydrides thereof can be used.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

In certain embodiments, the compound(s) described herein can exist as tautomers. All tautomers are included within the scope of the compounds presented herein.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

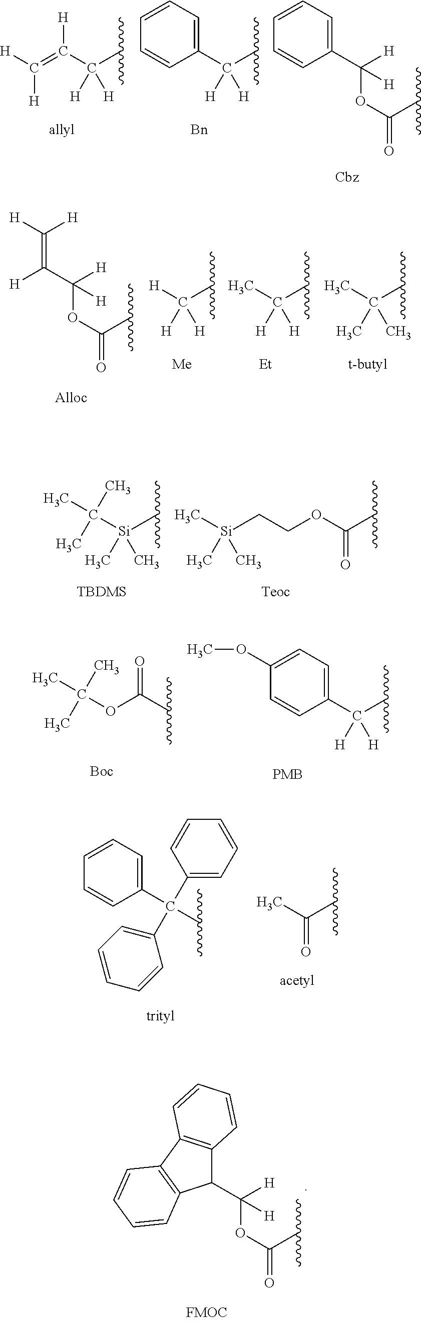

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Methods of Using Additives of Formula I

In various embodiments, a method of using a dental composition is provided. The method includes
applying a dental composition to an oral surface to form a coated oral surface, wherein the dental composition comprises a dental adhesive or resin and at least one compound of formula I,

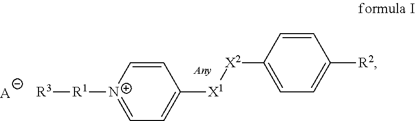

wherein:
A⁻ is an anion;
X¹ and X² are each independently selected from the group consisting of O, N, C, CH, and CH₂;
[symbol] represents a single, double, or triple bond;
R¹ is selected from the group consisting of —CₙH₍₂ₙ₊₁₎ and —CₙH₍₂ₙ₋₁₎;
R² is selected from the group consisting of —OC₂₋₈ alkenyl, methacrylate, acrylate, styrene, vinyl benzyl, isobutyrate, and —OH;
n is 2, 3, 4, 5, 6, 7, 8, 9, 10, 11, 12, 13, 14, 15, 16, 17, 18, 19, or 20;
wherein the composition optionally comprises at least one third moiety R³ attached to R¹, wherein R³ is selected from the group consisting of methacrylate, acrylate, styrene, vinyl benzyl, F, Cl, Br, and I;
wherein the compound of formula I is present in an amount of 0.1 to 1.25% (w/w) relative to the weight of the dental composition;
and
curing the coated oral surface.

In various embodiments, the oral surface is a tooth surface. In various embodiments, the oral surface is a dental article selected from the group consisting of crowns, bridges, veneers, inlays, onlays, fillings, and orthodontic appliances. In various embodiments, the oral surface includes an orthodontic appliance. The orthodontic appliance can have a base for receiving adhesive and it can include a flange made of metal, plastic, ceramic, or combinations thereof. Alternatively, the base can be a custom base formed from cured adhesive layer(s) (i.e. single or multi-layer adhesives). In various embodiments, the orthodontic appliance is selected from the group consisting of orthodontic brackets, buccal tubes, lingual retainers, orthodontic bands, bite openers, buttons, and cleats.

The dental compositions described herein can be applied to oral surfaces, dental articles, and/or orthodontal appliances using conventional dental techniques known in the art. The dental compositions can be cured by any suitable thermal or photochemical means

### EXAMPLES

Various embodiments of the present application can be better understood by reference to the following Examples which are offered by way of illustration. The scope of the present application is not limited to the Examples given herein. The enzymatic challenge measurement method described herein is described in Wang X, Song S, Chen L, Stafford CM, Sun J, Acta Biomater., 2018, 74, 326, and Yamauchi S, Wang X, Egusa H, Sun J. “High-performance dental adhesives containing an ether-based monomer” Journal of Dental Research, 2020, 99, 189, which are both incorporated by reference herein in their entireties.

### Synthesis

### General Information for Synthesis and Characterization

Commercially available materials purchased from Alfa Aesar (Tewksbury, Mass., USA), Sigma-Aldrich (Saint Louis, Mo., USA) and TCI America (Portland, Oreg., USA) were used as received. Proton and carbon nuclear magnetic resonance (¹H and ¹³C NMR) spectra were recorded on a Bruker instrument (600 MHz, Billerica, Mass., USA) using 5 mm tubes. Chemical shifts were recorded in parts per million (ppm, δ) relative to tetramethylsilane (δ=0.00), dimethylsulfoxide (δ=2.50) or chloroform (δ=7.26). ¹H NMR splitting patterns are designated as singlet (s), doublet (d), triplet (t), quartet (q), dd (doublet of doublets), and m (multiplets). High-resolution mass spectra (MS) were recorded on a JEOL AccuTOF (Peabody, Mass., USA) for ESI-TOF-MS analysis.

### Example 1: Synthesis of (E)-1-hexadecyl-4-((4-(methacryloyloxy)phenyl)diazenyl)-pyridinium bromide (Azo-QPS-C16)

Azo-QPS-C16 can be synthesized according to the synthetic procedure shown in FIG. TA. Five (5) g (53.2 mmol) of phenol and 4 g (60 mmol) of sodium nitrite were dissolved in 20 mL (10 wt %.) sodium hydroxide aqueous solution, and the mixture was stirred in an ice-bath at 0-4° C. The mixture was added dropwise to a pre-cooled solution made from 6 g (63.8 mmol) of 4-Aminopyridine in a hydrogen chloride aqueous solution. The reaction was stirred in the ice-bath for 30 minutes, and then stirred at room temperature overnight. The pH of the reaction was adjusted to 6-7 with ten percent by weight (10 wt %) sodium hydroxide; the precipitation was collected by filtration, and dried in air. The azo product was used in the next step without further purification.

In this next step, 4 g (20.1 mmol) of the azo and 1.25 equivalent of trimethylamine was dissolved in tetrahydrofuran (THF). One (1) equivalent of methacryloyl chloride was added to the reaction dropwise. The reaction was stirred at room temperature for 2 hours. The Azo-QPS-methacrylate compound was purified by column chromatography with an 87% yield.

Finally, the Azo-QPS-methacrylate compound was refluxed with 1.5 equivalent of desired alkyl bromide in acetonitrile for three days. The resulting dark red product was further purified by recrystallization with ether and acetone, affording 48-56% yield of Azo-QPS-C16. Chemical shift of protons in ¹H NMR (600 MHz, CDCl₃) spectrum follows: δ 9.64 (d, J=6.0 Hz, 2H), 8.30 (d, J=6.0 Hz, 2H), 8.10 (d, J=6.0, 11.0 Hz, 2H), 7.40 (d, J=16.0, 2 H), 6.42 (s, 1H), 5.86 (s, 1H), 5.11 (t, J=7.4 Hz, 2H), 2.08 (m, 5H), 1.33 (m, 26H), 0.88 (m, 3H) ppm; ¹³C NMR (600 MHz, CDCl₃) δ 165.01, 160.42, 156.39, 149.86, 147.21, 135.34, 128.44, 126.27, 123.07, 120.50, 62.03, 32.11, 31.93, 31.17, 29.70, 29.69, 29.66, 29.64, 29.60, 29.51, 29.37, 29.10, 28.77, 26.15, 22.70, 18.32, 14.12 ppm. Hi-Res MS (ESI): m/z calcd. for C₃₁H₄₆N₃O₂+, 492.3585; found [M]+: C₃₁H₄₆N₃O₂+, 492.3599.

### Example 2: Synthesis of MA-C6-Azo-Py-iBu

The first step of the synthesis is analogous to Example 1 in the synthesis for Azo-QPS-C16. In the next step, 4 g (20.1 mmol) of the azo-Py-OH and 1.25 equivalent of trimethylamine was dissolved in tetrahydrofuran (THF). One (1) equivalent of Isobutyryl chloride (Sigma-Aldrich, Saint Louis, Mo., USA) was added to the reaction dropwise. The reaction was stirred at room temperature for 2 hours. The Azo-Py-iBu compound was purified by column chromatography with an 80% yield. In the last step to synthesize MA-C6-Azo-Py-iBu, a round bottom flask equipped with a magnetic stir bar was charged with (E)-4-(pyridin-4-yldiazenyl)phenyl isobutyrate (Azo-Py-iBu, 0.54 g, 2.00 mmol, 1.00 equivalent) and 6-bromohexyl 2-methylprop-2-enoate (0.75 g, 3.00 mmol, 1.50 equivalent, Synnovator Inc. Durham, NC, USA) and dissolved in acetonitrile (50.0 mL, 0.04 M). The flask was equipped with a reflux condenser, and the reaction was allowed to stir for 3 days at 80° C. After this period, the solvent was removed in vacuo, and the reaction mixture was dissolved in acetone and precipitated in diethyl ether to yield an orange powder. (0.7 g, 68% yield)¹H NMR (DMSO-d6) δ:1.29 (6H, d, J=7.0 Hz, CH₃), 1.40 (4H, m, CH₂), 1.65 (2H, m, CH₂), 1.88 (3H, s, CH₃—C═C), 1.99 (2H, m, CH₂), 2.91 (1H, m, CH), 4.11 (2H, t, J=6.5 Hz, CH₂—O), 4.69 (2H, t, J=7.4 Hz, CH₂—N), 5.68 (1H, s, C═C), 6.02 (1H, s, C═C), 7.51 (2H, d, J=8.8 Hz, aromatic), 8.15 (2H, d, J=8.8 Hz, aromatic), 8.43 (2H, d, J=6.8 Hz, aromatic), 9.33 (2H, d, J=6.8 Hz, aromatic); ¹³C NMR (DMSO-d6) δ: 18.48 (1C, CH₃—C═C), 19.03 (2C, CH₃), 25.35, 25.49, 28.30, 31.10 (4C, CH₂), 33.91 (1C, CH), 60.95 (1C, CH₂—N), 64.59 (1C, CH₂-0), 120.60, 123.96, 126.09, 147.79 (8C, aromatic), 126.05, 136.43 (2C, C═C), 150.00, 155.86, 160.68 (3C, C—N═N, C—O), 167.03, 175.07 (2C, C═O).

Compositions of Compounds of Formula I and Dental Adhesives and/or Resin

Mixtures of compounds of formula I and resins or adhesives are formed by mixing different weights of the components and then stirring at 200 rpm by using a magnetic stir-bar for 24 hours at room temperature. All mass measurements were performed using a high precision balance (Sartorius BP211D, Sartorius AG, Gottingen, Germany) with a resolution/range of 0.01 mg/80 g. In one example, 4.97 mg Azo-QPS-C16 was added into 331.60 mg SB adhesive, producing the adhesive including the additive at approximately 1.48 wt %. Other dental materials with controlled wt % of additive were also prepared by this method.

Additives of formula I, including Azo-QPS-C16, have been added into multiple adhesives, including an experimental adhesive containing 60 wt % of 2-bis(4-(2-hydroxy-3-methacryl-oxypropoxy)-phenyl)-propane) (Bis-GMA) and 40 wt % of 2-hydroxyethyl-methacrylate (HEMA) (referred to as B6H4), a BisGMA/HEMA-based commercial adhesive, 3MT^{M} Adper™ Scotchbond™ Multi-Purpose Adhesive (SB) Prime & Bond NT Light-Cure (PBNT, Dentsply), Single Bond Plus: BisGMA/HEMA based; two-step adhesive (SBP, 5th generation, 3M), Scotchbond Universal Plus: BisGMA-free two-step adhesive (SUP, 5th generation, 3M), Adper™ Easy Bond Self-Etch Adhesive (EBSE, 7th generation, 3M) and Xeno® IV Adhesive (XIV, 7th generation, Dentsply). Additives were also added to BisGMA/TEGDMA resins.

The degree of conversion (DC) and glass transition temperature (T_{g}) of adhesives and resins with or without the additives (such as Azo-QPS-C16) were measured by Fourier transform infrared (FTIR) spectrometer and dynamic mechanical analyzer (DMA), respectively. The stability against enzymatic challenges of four adhesives (and resins) was evaluated using an atomic force microscopy (AFM)-based method. The adhesives' bonding strength and bonding durability are determined and compared based on shear bond strength (SBS), micro-tensile bond strength (pTBS), and bonding durability under the challenges of thermal cycling.

### Measurements and Results

### Degree of Conversion

The degree of conversion (DC) of the dental materials, which represents the proportion of polymerized monomers after setting (curing), was evaluated using a Thermo Nicolet Nexus 670 Fourier-transform infrared (FT-IR) spectrometer equipped with a KBr beamsplitter, an MCT/A detector and an attenuated total reflectance (ATR) accessory. The aromatic C═C absorption band at 1608 cm⁻¹ of Bis-GMA was used as the internal standard for the dental material formulations. To prepare samples for analysis, 20 micro-liter of the dental material mixture was dropped onto the top of the ATR crystal (diameter=2 mm). Then the top of the resin droplet was covered by Mylar film (15 mm*15 mm, thickness=50 μm). Then the dental material was cured for 20 seconds using a SmartLite® Max (Dentsply Caulk, Milford, DE, USA) LED curing light with 1 W/cm² intensity. The DC values were measured 30 min after light curing. DC was calculated according to the following equation:

$$DC ( \% ) = ( {A}_{1} / {A}_{0} - {A}_{1}^{′} / {A}_{0}^{′} ) / ( {A}_{1} / {A}_{0} ) \times 100 , \qquad ( 1 )$$

where A₀ is the area under the reference peak (1608 cm⁻¹) prior to polymerization, A₁ is the area under the peak for the sample (1638 cm⁻¹ for Azo-QPS-C16) prior to polymerization, A₀′ is the area under the reference peak after polymerization peak and A₁′ is the area under the sample peak after polymerization.

For other additive materials, the wavenumber for the sample peak in the FTIR may differ from that for Azo-QPS-C16, according to the bonding present in the structure. For example, if there is no aromatic C═C in the system, then a C═O peak, or N—H peak may also be used as a reference.

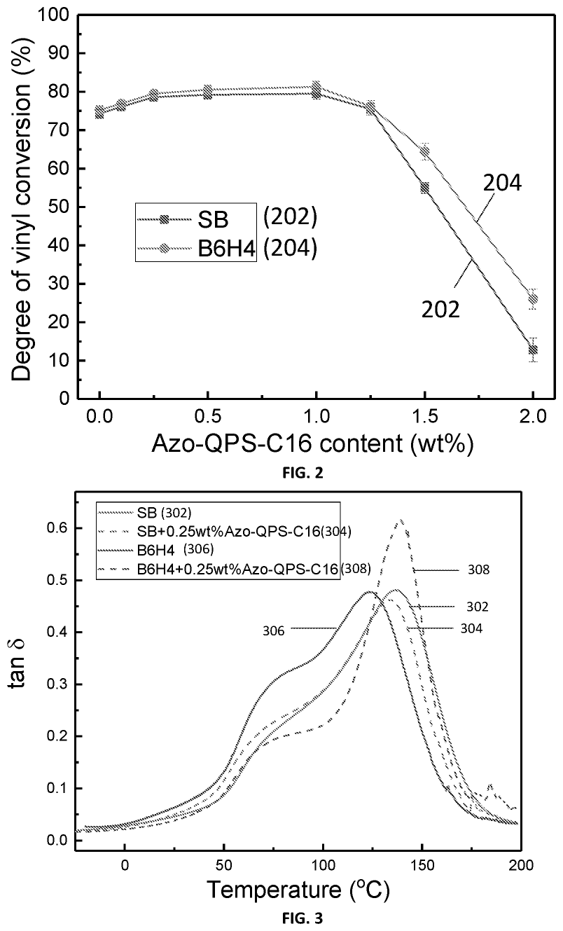

FIG. 2 is a graph showing the effect of an exemplary additive of the present invention on the degree of vinyl conversion (DC) of two adhesive materials, SB and an experimental adhesive (B6H4). A photoinitiator was added to the B6H4 adhesive. The initiator (CQ/4E) comprised 0.2 wt % camphorquinone (CQ; Aldrich, Saint Louis, MO, USA) and 0.8 wt % ethyl 4-N, N-dimethylaminobenzoate (4EDMAB, 4E; Aldrich, SaintLouis, MO, USA). The DC values for two adhesives, with an additive of Azo-QPS-C16 from no additive up to approximately 2.0 wt % additive are also listed in Table 1.

TABLE 1
Degrees of vinyl Conversion with Compositions Containing a
Dental Resin and/or Adhesive and an Additive of formula I.

| Resin (or adhesive) | | |
|---|---|---|
| Mixture | Additive (approx. wt %) | DC (%) |
| Scotchbond Multi- | None | 74.3 ± 1.2 |
| Purpose (SB) | 0.1 wt % Azo-QPS-C16 | 76.1 ± 0.8 |
| | 0.25 wt % Azo-QPS-C16 | 78.6 ± 0.9 |
| | 0.5 wt % Azo-QPS-C16 | 79.2 ± 1.1 |
| | 1.0 wt % Azo-QPS-C16 | 79.5 ± 1.4 |
| | 1.25 wt % Azo-QPS-C16 | 75.5 ± 1.5 |
| | 1.5 wt % Azo-QPS-C16 | 55.0 ± 1.4 |
| | 2.0 wt % Azo-QPS-C16 | 12.8 ± 3.1 |
| B6H4/CQ/4E | None | 75.2 ± 1.1 |
| | 0.1 wt % Azo-QPS-C16 | 76.9 ± 0.7 |
| | 0.25 wt % Azo-QPS-C16 | 79.5 ± 0.8 |
| | 0.5 wt % Azo-QPS-C16 | 80.5 ± 1.2 |
| | 1.0 wt % Azo-QPS-C16 | 81.3 ± 1.4 |
| | 1.25 wt % Azo-QPS-C16 | 76.2 ± 1.5 |
| | 1.5 wt % Azo-QPS-C16 | 64.4 ± 2.2 |
| | 2.0 wt % Azo-QPS-C16 | 26.0 ± 2.6 |

Adding as little as approximately 0.1 wt % of Azo-QPS-C16 enhances the DC of both adhesives, and the DC increases up to approximately 1 wt % inclusion of the additive. Compared to the adhesives without the additive, the DC remains higher for additive inclusion at up to about 1.25 wt %. As can be seen, adding more than approximately 1.25 wt % of Azo-QPS-C16, such as about 1.5 wt % or about 2 wt % leads to a significant DC decrease to values well below those for the adhesives without additives. In all experiments, the same polymerization conditions as described above were used.

Table 2 shows structures of azo-type quaternary pyridinium salts and the effect on the DC of a resin they are added to. The resin was prepared by mixing 70 wt % Bis-GMA with 30 wt % triethylene glycol dimethacrylate (TEGDMA), adding an initiator comprising 0.2 wt % camphorquinone (CQ; Aldrich, Saint Louis, MO, USA) and 0.8 wt % ethyl 4-N, N-dimethylaminobenzoate (4EDMAB; Aldrich, SaintLouis, MO, USA) (CQ/4E), and including 0.5 wt % of an Azo compound as an additive.

Samples were prepared and exposed to light according to the conditions described above. The first four compounds show a statistically significant improvement in DC, whereas the 5^{th} compound shows a very small change that is within experimental measurement error. Thus not all azo-type quaternary pyridinium salts contribute to increased DC.

TABLE 2
Effect of Additives of formula I on Degree of Conversion

| Compound | | Degree of |
|---|---|---|
| Name | Structure | Conversion |
| Azo-QPS- C16-MA | 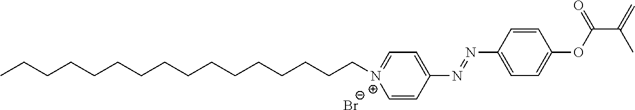 [SMILES: C=C(C)C(=O)Oc1ccc(/N=N/c2cc[n+](CCCCCCCCCCCCCCCC)cc2)cc1 ; [Br-]] | 74.5% (increase of 4.3%) |
| Azo-QPS- C2-MA | 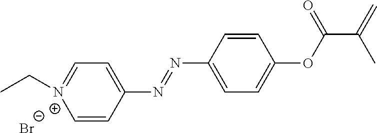 [SMILES: C=C(C)C(=O)Oc1ccc(/N=N/c2cc[n+](CC)cc2)cc1 ; [Br-]] | 72.3% (increase of 2.1%) |
| MA-C6- Azo-Py-iBu | 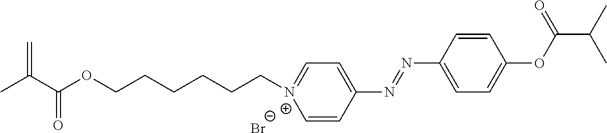 [SMILES: C=C(C)C(=O)OCCCCCC[n+]1ccc(/N=N/c2ccc(OC(=O)C(C)C)cc2)cc1 ; [Br-]] | 74.9% (increase of 4.7%) |
| Br-C6-Azo- Py-iBu | 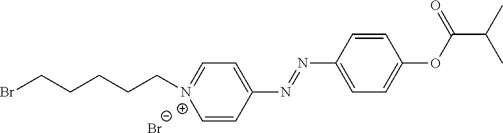 [SMILES: CC(C)C(=O)Oc1ccc(/N=N/c2cc[n+](CCCCCBr)cc2)cc1 ; [Br-]] | 75.1% (increase of 4.9%) |
| MA-CB13- Azo-QPS- iBu | 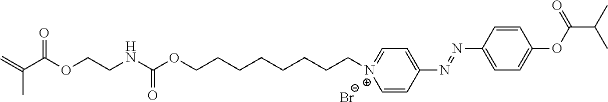 [SMILES: C=C(C)C(=O)OCCNC(=O)OCCCCCCCC[n+]1ccc(/N=N/c2ccc(OC(=O)C(C)C)cc2)cc1 ; [Br-]] | 70.7% (increase of 0.5%) |

Other additives, either Azo-pyridine compounds or azo-benzene compounds with and without side groups, were also evaluated. As shown in Table 3, these additives either did not change the DC or reduced the DC.

TABLE 3
Degree of Conversion with Other Azo-pyridine or Azo-benzene Compounds

| Compound Name | Structure | Degree of Conversion |
|---|---|---|
| Azo-Py-MA | 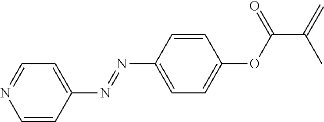 [SMILES: C=C(C)C(=O)Oc1ccc(/N=N/c2ccncc2)cc1] | 70.2% (no change) |
| Azo-Py-iBu | 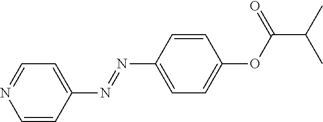 [SMILES: CC(C)C(=O)Oc1ccc(/N=N/c2ccncc2)cc1] | 66.7% (decrease by 3.5%) |
| Azo-Py-OH | 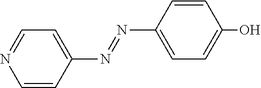 [SMILES: Oc1ccc(/N=N/c2ccncc2)cc1] | 68.4% (decrease by 1.8%) |
| | 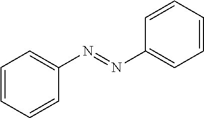 [SMILES: c1ccc(/N=N/c2ccccc2)cc1] | No change |
| | 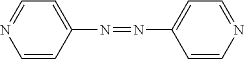 [SMILES: c1cc(N=Nc2ccncc2)ccn1] | No change |
| | 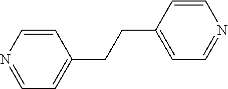 [SMILES: c1cc(CCc2ccncc2)ccn1] | No change |

Table 4 shows the degree of conversion for 0.25 wt % Azo-QPS-C16 added to PBNT, SBP, SUP, EBSE and XIV adhesives.

TABLE 4

| | Degree of | Degree of Conversion (%) |
|---|---|---|
| Adhesive/ | Conversion (%) | (approx 0.25 wt % |
| Resin | (no Azo-QPS-C16) | Azo-QPS-C16) |
| PBNT | 62.8 ± 1.3 | 66.4 ± 0.7 |
| SBP | 65.3 ± 0.8 | 70.3 ± 0.5 |
| SUP | 67.6 ± 0.6 | 72.5 ± 1.0 |
| EBSE | 67.5 ± 0.8 | 73.7 ± 0.6 |
| XIV | 70.3 ± 0.9 | 74.2 ± 1.1 |

These results show only certain azo-type quaternary pyridinium salts can enhance the DC of dental resins and adhesives, and only when incorporated into the resin or adhesive over a small range (wt % o) of up to about 1.25%.

### Glass Transition Temperature

The glass transition temperature (Tg) of adhesives with or without additive were measured by dynamic mechanical analysis (DMA). FIG. 3 shows the effect on Tg of two adhesives, SB and B6H4, by adding an exemplary additive of the present invention, Azo-QPS-C16 at approximately 0.25 wt %.

For B6H4, the glass transition temperature increases while the tan δ peak also narrowed. For SB, the tan δ peak narrowed. Narrowing of the tan δ peak indicates an increased crosslink density within the polymer (i.e. denser crosslinking), which can lead to enhanced stability.

### Biostability

Biostability of the dental materials such as resins and adhesives is of critical importance to their durability. These materials may break down in the oral environment, which may be due to enzymatic and/or bacterial challenges, which can result in failure of the restoration and secondary caries. The stability against enzymatic challenges of dental materials including adhesives and resins, with and without additives, was evaluated using an atomic force microscopy (AFM)-based method, described by Wang et al., in “Short-time dental resin biostability and kinetics of enzymatic degradation”, Acta Biomaterialia, Volume 74, 1 Jul. 2018, Pages 326-333, incorporated herein in its entirety. Briefly, this method allows evaluation of materials by assessing the step height (H) and the changes in the step height (ΔH), that are caused by enzymatic exposure. The ΔH values are caused as material exposed to salivary enzymes or the like is removed due to degradation of the exposed material.

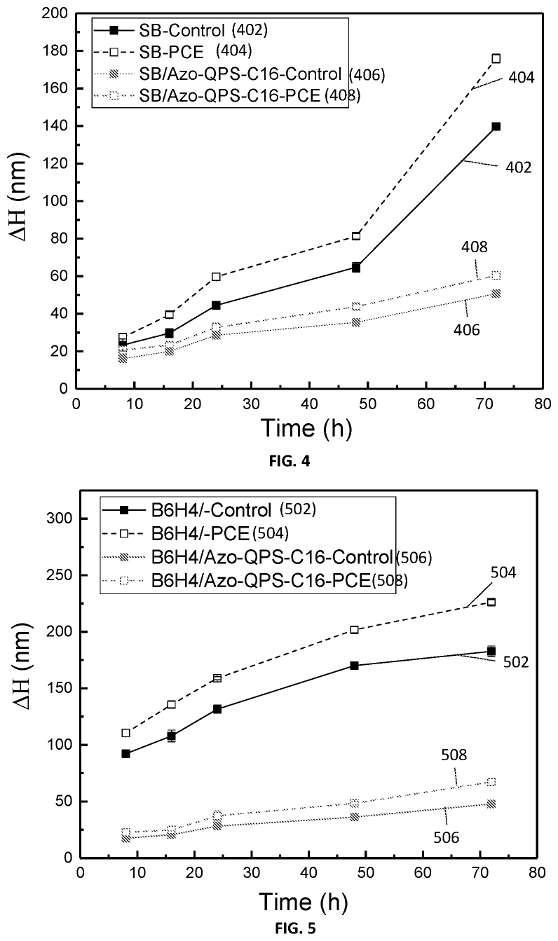

The stability of SB was compared to the same adhesive incorporating Azo-QPS-C16 as an additive at approximately 0.25 wt %. Samples were prepared according to the method described by Wang, and were exposed to either pseudocholine esterase from equine serum (PCE, Product No. C₇₅₁₂, Sigma, Saint Louis, MO, USA) were reconstituted in saline solution containing 40 mM magnesium chloride and 99 mM of sodium chloride following vender's instruction, or a control solution without PCE for various times up to 72 hours. Measurement results for ΔH as a function of time are shown in FIG. 4. The SB control sample exhibited a ΔH of approximately 140 nm after 72 hours. For the SB sample exposed to PCE, the ΔH value increased to about 175 nm. In comparison, the addition of approximately 0.25 wt % Azo-QPS-C16 reduced the ΔH values after 72 hours to about 50 nm and about 60 nm for the control and PCE-exposed samples, respectively, thus showing inclusion of Azo-QPC-C16 in the adhesive improved the stability dramatically.

Similarly, FIG. 5 shows the effect of adding Azo-QPS-C16 at 0.25 wt % to the B6H4 adhesive, which also included a photoinitiator (CQ/4E). The control sample without any additive exhibited a ΔH value of about 180 nm, while the sample exposed to PCE exhibited a ΔH value of about 225 nm after 72 hours. Samples including approximately 0.25 wt % Azo-QPS-C16 in the adhesive exhibited ΔH values of about 50 nm (control) and about 70 nm (exposed to PCE), clearly showing inclusion of Azo-QPC-C16 in the adhesive improved the stability dramatically.

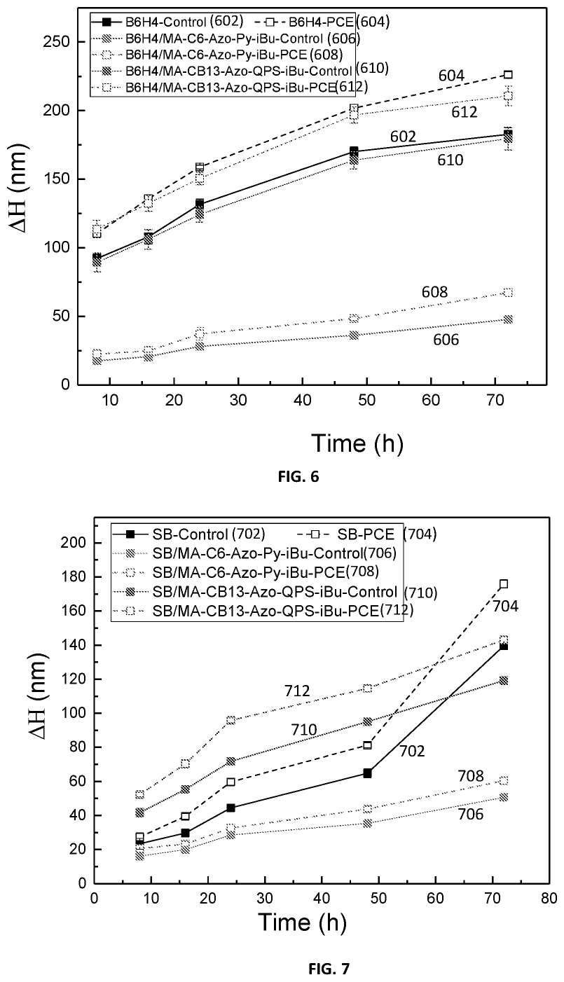

FIG. 6 compares the effect of adding MA-CB13-Azo-QPS-iBu and MA-C6-Azo-Py-iBu to B6H4 on the stability. The additives were included at approximately 0.25 wt %, and FIG. 7 compares the effects of adding MA-CB13-Azo-QPS-iBu and MA-C6-Azo-Py-iBu to SB on the stability. From FIG. 6 it can be seen that whereas MA-CB13-Azo-QPS-iBu does not decrease the ΔH value for the control and PCE-exposed samples (values between about 175 nm and 225 nm, adding MA-C6-Azo-Py-iBu reduces the ΔH value to approximately 50 nm (control) and 65 nm (PCE). For Scotchbond, as shown in FIG. 7, adding MA-C6-Azo-Py-iBu reduces the ΔH value to approximately 50 nm (control) and 70 nm (PCE), whereas samples with no additive or MA-CB13-Azo-QPS-iBu have higher ΔH values between about 120 nm and 180 nm.

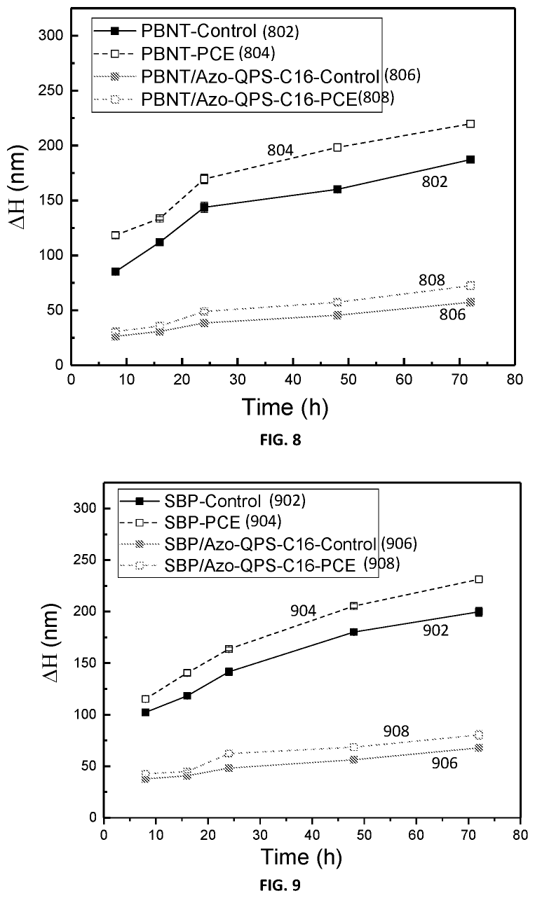

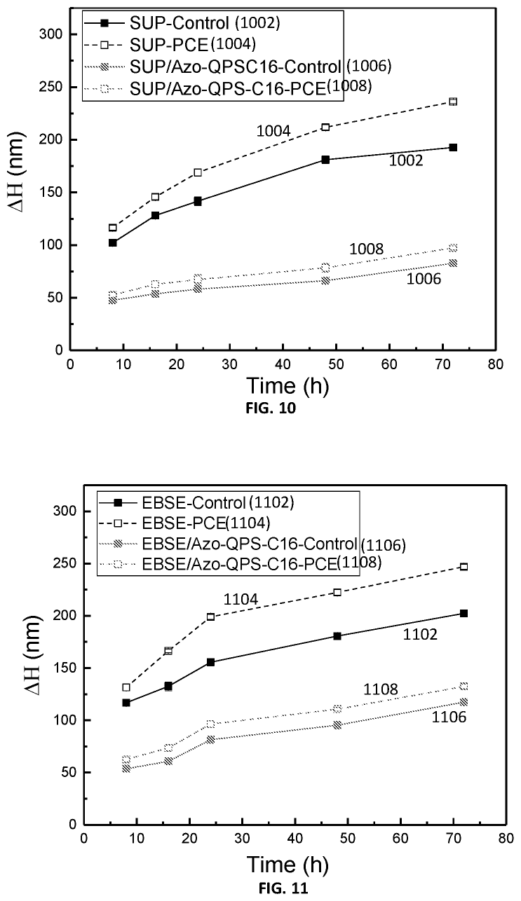

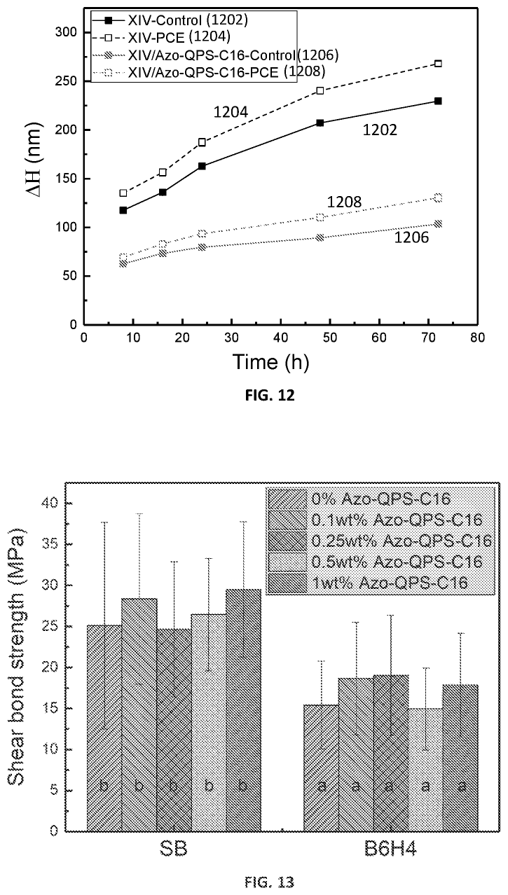

Azo-QPS-C16 was also added to adhesives PBNT, SBP, SUP, EBSE and XIV. FIGS. 8-12 show the effects on the stabilities, as measured by ΔH values, for adhesives with approximately 0.25 wt % additive, and no additive. In all cases, the addition of Azo-QPS-C16 decreased the ΔH values, indicating that it successfully increased the stability of the adhesive against enzymatic challenge.

Adding MA-CB13-Azo-QPS-iBu to dental adhesives, on the other hand, has not been demonstrated to improve the biostability of the adhesives.

### Bonding Strength and Bonding Durability

The adhesives' bonding strength and bonding durability are determined and compared based on shear bond strength (SBS), micro-tensile bond strength (pTBS), and bonding durability under the challenges of thermal cycling. The methods for measuring SBS, μTBS, and for carrying out thermal cycling are described by Yamauchi et al., in “High-performance dental adhesives containing an ether-based monomer,” Journal of Dental Research, 2020, 99, 189, and the supplementary information associated with the publication, both of which are incorporated herein by reference.

### Shear Bond Strength (SBS) Measurements

Briefly, teeth were embedded with Fastray™ composite (Harry J. Bosworth Company, Skokie, IL, USA) in cylindrical holders and ground perpendicular to their long axis with 400-grit SiC paper until the occlusal enamel was completely removed. A three-step adhesive procedure entailed: 1) etching of dentin surface with a 32% (by mass) phosphoric acid gel (Scotchbond™ Universal Etchant; 3M ESPE., Seefeld, Germany) for 15 s and rinsing with distilled water (after rinsing, dentin surface was kept hydrated with a moist blotting paper); 2) applying primer by brushing on the dentin surface, accumulating 5 layers, air drying between layers to evaporate the solvent; then 3) applying bonding agents by brushing once on the primed dentin surface. The entire dentin surface was then light-cured for 10 s (for adhesives) or 60 s (for dental composites) with the use of an 8 mm tip on a quartz halogen light source having 550 mW/cm² intensity (Spectrum 800, Caulk/Dentsply, Milford, DE, USA). A poly(tetrafluoroethylene)-covered stainless steel ring (opening diameter 4 mm; thickness 1.5 mm) defined the bonding area through which the composite was applied onto the coated dentin. The ring was held down by a polycarbonate holder, and the iris was filled with the under dental material under test. The entire assembly was placed in distilled water for 4 min after 1-min light irradiation and stored for 24 h at room temperature before conducting a bond test in the shear mode. SB was used as the commercial control. The ring and the composite were sheared off, at a crosshead speed of 0.5 mm/min, with a flat chisel pressing against the edge of the steel iris. The flat chisel was controlled by a Universal Testing Machine (Instron 5500R, Instron Corp., Canton, MA, USA). The maximum load was converted into the SBS by following this relationship, SBS=loadmax/area, where the area is the contacting area between composites and adhesives defined by the inside diameter (approx. 4 mm) of the stainless ring. The mean values of SBS were the average of five measurements for each composition.

### Microtensile Bond Strength (μTBS)

Teeth were embedded with Fastray™ composite (Harry J. Bosworth Company, Skokie, IL, USA) in metal cubic holders, then ground perpendicular to their long axis with 400-grit SiC paper until the occlusal enamel was completely removed. The bonding procedures were the same as described in SBS tests. After applying bonding agent, a commercial composite (Filtek™ Z250 Universal Restorative, 3M ESPE, St. Paul, MN, USA) was added in four 1 mm thick increments. Specimens were light-cured for 1 min using a quartz halogen light source at 550 mW/cm² (Spectrum 800, Caulk/Dentsply, Milford, DE, USA) and then stored in distilled water for 24 h. Following the water storage, specimens were cut in mesiodistal direction and parallel to the horizontal plane of the teeth using a 0.2 mm diamond disk (IsoMet™ Diamond Wafering Blades, Buehler, Lake Bluff, IL, USA) at 100 rpm speed under running water. Sample preparation is described by Wang et al., in “Improve Dentin Bonding Performance Using a Hydrolytically Stable, Ether-Based Primer”, J. Funct. Biomater. 2022, 13, 128, and the supplementary materials, both of which are incorporated by reference herein in their entireties The resulting 0.7-1.0 mm thick by 0.7-1.0 mm wide beams were divided into two groups: one group was examined immediately after sectioning, and the other group was assessed after thermal cycling. The beams were glued to a μTBS testing device using Zapit® (Dental Ventures of America, Corona, CA, USA). Tests were performed by using a Universal Testing Machine (Instron 5500R, Instron Corp., Canton, MA, USA) under tension at 1 mm/min until failure. The TBS values (MPa) were calculated by dividing the load at failure by the cross-sectional bonding area. The TBS values are reported as an average of at least 15 measurements using beams from at least three different teeth.

In some embodiments, the samples are in a rectangular prism shape that we cut in two directions for defining the base area (width and thickness) while no cut is in the length direction. The sample preparation is illustrated in the Figure S3 of the publication J. Funct. Biomater. 2022, 13(3), 128.

### Thermal Cycling for Adhesive Durability Evaluation

The thermal cycling was done after the teeth were sectioned into beams. The beams for each test (each resin composition before or after thermal cycling) were originated from 3-4 different teeth. Thermocycling tests were performed between 5° C. and 55° C. on beams prepared for TBS tests using a thermocycling device described by Xu et al., in “Effect of thermal cycling on whisker-reinforced dental resin composites”, J Mater Sci-Mater Med. 13(9):875-883 (2002). The temperatures were maintained by two water tanks. Specimens were switched between tanks with the dwell time in each tank for 30 s. The transferring time between tanks was 10 s. After the completion of 10,000 thermal cycles, and 20,000 thermal cycles, the average μTBS of each adhesive was determined and compared with the TBS value before cycling. The TBS values are reported as an average of at least 15 measurements using beams from at least three different teeth.

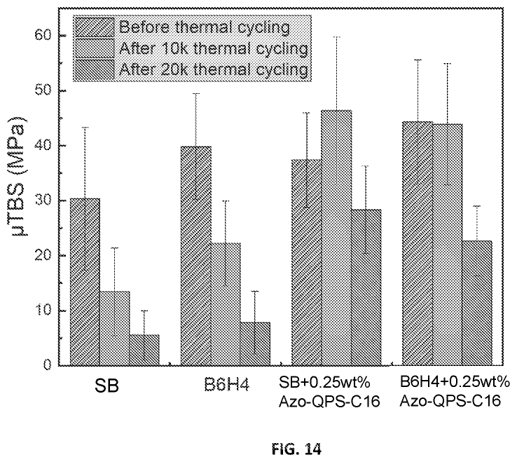

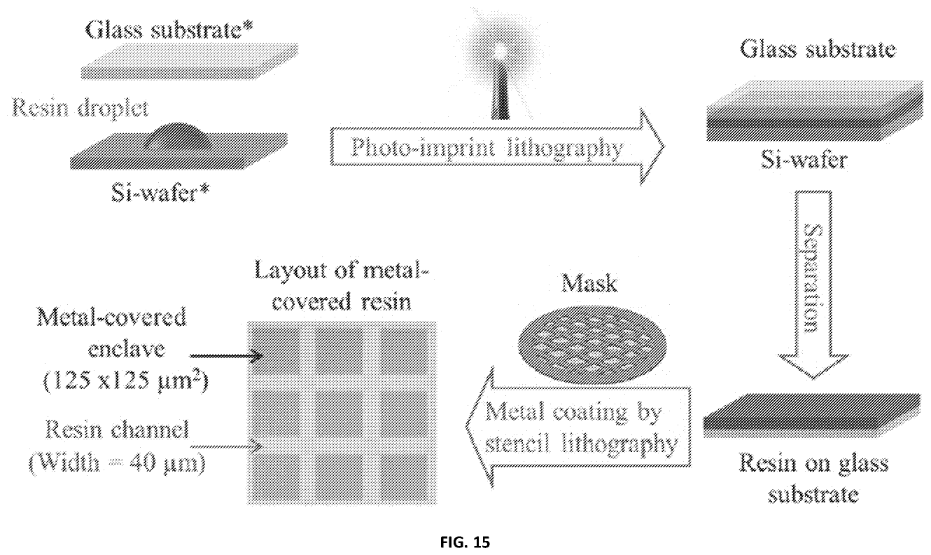

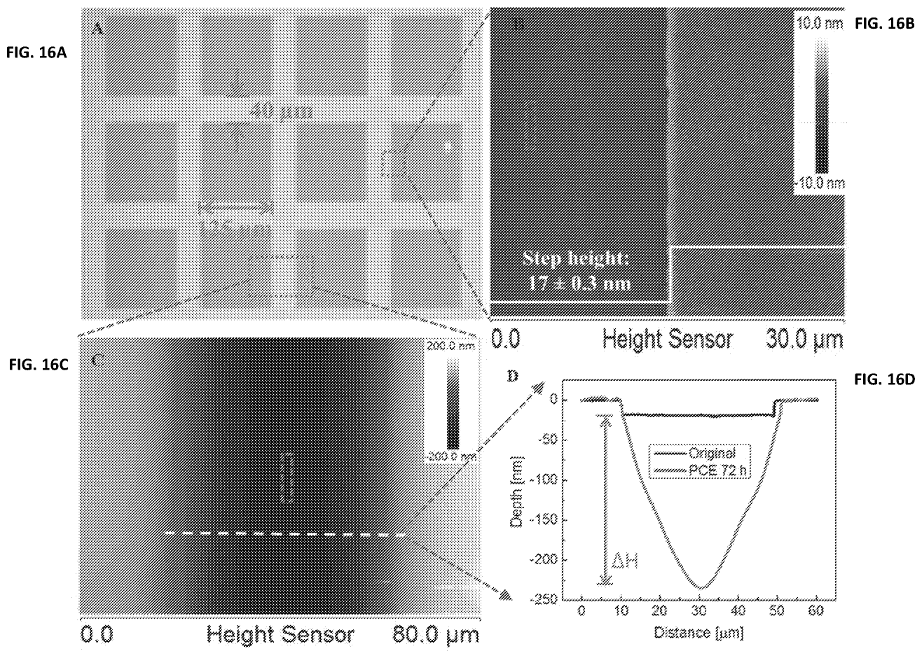

The shear bond strength was measured for both SB and B6H4 adhesives, with and without Azo-QPS-C16 additives. As shown in FIG. 13, for both adhesives, adding Azo-QPS-C16 at levels up to approximately 1.0 wt % did not affect the shear bond strength of the adhesive.

Durability of the adhesive bonding to dentin was assessed by thermal cycling of the materials, followed by measurement of the microtensile bond strength (μTBS). Results for SB and B6H4 are shown in FIG. 14, along with comparison data for these adhesives where about 0.25 wt % Azo-QPS-C16 was incorporated. The μTBS was measured prior to temperature cycling, after 10,000 cycles and after 20,000 cycles. After 10,000 cycles, the μTBS for the adhesives alone approximately halved, and decreased even further after 20,000 cycles. For the adhesive samples that included Azo-QPS-C16, after 10,000 cycles, the μTBS remained unaffected. Even after 20,000 cycles, although they had started to decrease, their values were still more than half the starting values, and in comparison with the samples without the additives, the μTBS values remained considerably higher, indicating the compound as an additive can enhance the bonding durability of the adhesive material.

Azo-QPS-C16 was also added to five other adhesives, and the shear bond strength was measured. The values are shown in Table 5. For all materials, the SBS values were not compromised by addition of the Azo-QPS-C16.

TABLE 5
Measured SBS Values with Azo-QPS-
C16 and Dental Adhesives/Resins

| Adhesive/ | SBS (MPa) | SBS (MPa) (approx. |
|---|---|---|
| Resin | (No Azo-QPS-C16) | 0.25 wt % Azo-QPS-C16) |
| PBNT | 26.7 ± 5.3 | 24.2 ± 4.5 |
| SBP | 19.4 ± 3.7 | 21.1 ± 4.4 |
| SUP | 20.6 ± 4.3 | 22.5 ± 3.8 |
| EBSE | 20.4 ± 4.3 | 24.2 ± 5.9 |
| XIV | 23.4 ± 7.1 | 19.3 ± 6.4 |

Thus, additives of the present disclosure, when added to adhesives or resins at a low wt % less than or equal to approximately 1.25% can enhance the DC, T_{g} and stability against enzymatic degradation for dental materials such as dental adhesives and dental resins.

In one non-limiting example, enhancements in DC, T_{g}, and the stability against enzymatic degradation for both experimental and commercial adhesives upon adding approximately 0.25-0.5 wt % of Azo-QPS-C16 have shown by FTIR, DMA, and the AFM-based evaluation method, respectively. Both experimental and commercial adhesives with Azo-QPS-C16 achieved equivalent SBS (>15 MPa) and μTBS (>35 MPa) to their counterparts without Azo-QPS-C16. After 10,000 thermal cycles, both adhesives without Azo-QPS-C16 failed, while TBS values of the two adhesives with Azo-QPS-C16 remained unchanged. Azo-QPS-C16 is shown to be an effective additive for dental adhesives to enhance DC, T_{g}, and stability against enzymatic degradation. Furthermore, adding Azo-QPS-C16 can enhance the durability of dentin bonding without jeopardizing bonding strength. The current results suggest significant potential for further developing dental restorative materials with extended service life.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### ENUMERATED EMBODIMENTS

The following enumerated embodiments are provided, the numbering of which is not to be construed as designating levels of importance:

Embodiment 1 provides dental composition comprising:
a dental adhesive or resin, and
at least one compound of formula I,

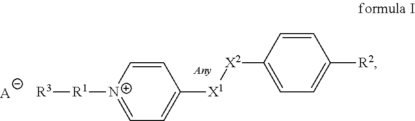

wherein:
   A⁻ is an anion;
   X¹ and X² are each independently selected from the group consisting of O, N, C, CH, and CH₂;
   [symbol] represents a single, double, or triple bond;
   R¹ is selected from the group consisting of —CₙH₍₂ₙ₊₁₎ and —CₙH₍₂ₙ₋₁₎;
   R² is selected from the group consisting of —OC₂₋₈ alkenyl, methacrylate, acrylate, styrene, vinyl benzyl, isobutyrate, and —OH;
   n is 2, 3, 4, 5, 6, 7, 8, 9, 10, 11, 12, 13, 14, 15, 16, 17, 18, 19, or 20;
   wherein the composition optionally comprises at least one third moiety R³ attached to R¹, wherein R³ is selected from the group consisting of methacrylate, acrylate, styrene, vinyl benzyl, F, Cl, Br, and I; and
   wherein the compound of formula I is present in an amount of 0.1 to 1.25% (w/w) relative to the weight of the dental composition.

Embodiment 2 provides the dental composition of embodiment 1, wherein [symbol] represents a double bond.

Embodiment 3 provides the dental composition of any one of embodiments 1-2, wherein X¹ is N and X² is N.

Embodiment 4 provides the dental composition of any one of embodiments 1-3, wherein A⁻ is selected from the group consisting of NO₃⁻, NO₂⁻, SCN⁻, CN⁻, PO₄³⁻, SO₄²⁻, F⁻, Cl⁻, Br⁻, I⁻, BF₄⁻, AsF₆⁻, SbF₆⁻, CH₃CO₂⁻, CF₃SO₃⁻, (CF₃SO₂)₂N⁻, (CF₃SO₂)₃C⁻, and CF₃COO⁻.

Embodiment 5 provides the dental composition of any one of embodiments 1-4, wherein A⁻ is Br⁻.

Embodiment 6 provides the dental composition of any one of embodiments 1-5, wherein the compound of formula I is present in an amount of 0.1 to 1.25% (w/w) relative to the weight of the dental composition.

Embodiment 7 provides the dental composition of any one of embodiments 1-6, wherein the compound of formula I is present in an amount of 0.2 to 1% (w/w) relative to the weight of the dental composition.

Embodiment 8 provides the dental composition of any one of embodiments 1-7, wherein the compound of formula I is selected from the group consisting of.

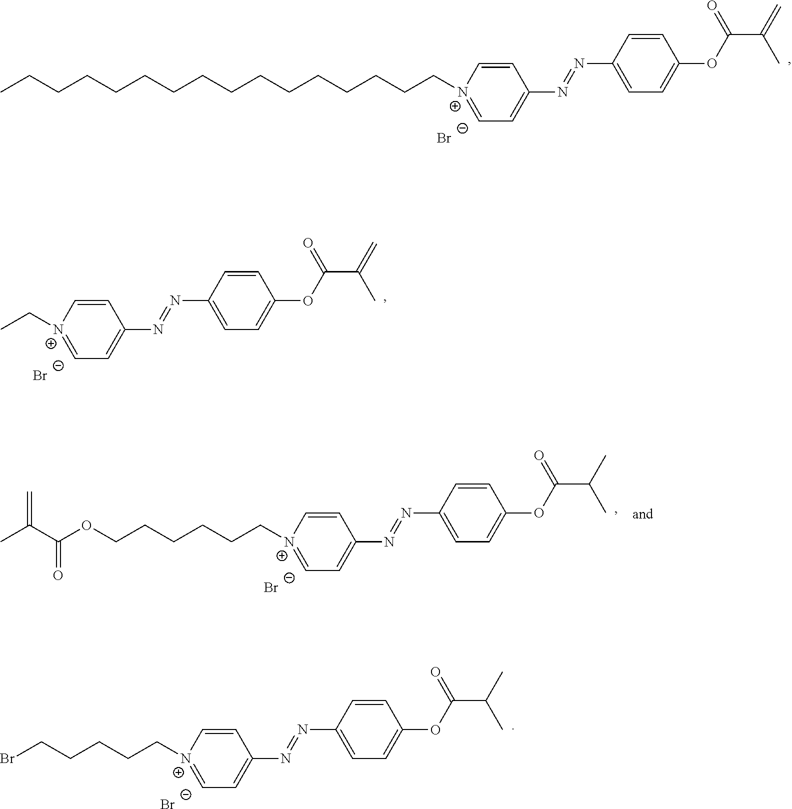

Embodiment 9 provides a cured composition comprising a polymerized product of a dental adhesive or resin and at least one compound of formula I,

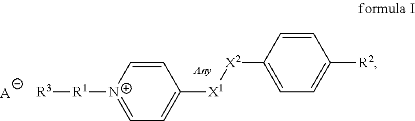

wherein:
A⁻ is an anion;
X¹ and X² are each independently selected from the group consisting of O, N, C, CH, and CH₂;
[symbol] represents a single, double, or triple bond;
R¹ is selected from the group consisting of —CₙH₍₂ₙ₊₁₎ and —CₙH₍₂ₙ₋₁₎;
R² is selected from the group consisting of —OC₂₋₈ alkenyl, methacrylate, acrylate, styrene, vinyl benzyl, isobutyrate, and —OH;
n is 2,3,4,5, 6, 7, 8, 9, 10, 11, 12, 13, 14, 15, 16, 17, 18, 19, or 20;
wherein the composition optionally comprises at least one third moiety R³ attached to R¹, wherein R³ is selected from the group consisting of methacrylate, acrylate, styrene, vinyl benzyl, F, Cl, Br. and I; and
wherein the compound of formula I is present in an amount of 0.1 to 1.25% (w/w) relative to the weight of the dental composition.

Embodiment 10 provides the cured composition of embodiment 9, wherein the polymerized product has a higher degree of vinyl bond as compared to an equivalent cured composition lacking the compound of formula I.

Embodiment 11 provides the cured composition of any one of embodiments 9-10, wherein the polymerized product has a higher biostability as compared to an equivalent cured composition lacking the compound of formula I.

Embodiment 12 provides the cured composition of any one of embodiments 9-11, wherein polymerized product has a higher durability as compared to an equivalent cured composition lacking the compound of formula I.

Embodiment 13 provides the cured composition of any one of embodiments 9-12, wherein [symbol] represents a double bond.

Embodiment 14 provides the cured composition of any one of embodiments 9-13, wherein X¹ is N and X² is N.

Embodiment 15 provides the cured composition of any one of embodiments 9-14, wherein A⁻ is selected from the group consisting of NO₃⁻, NO₂⁻, SCN⁻, CN⁻, PO₄³⁻, SO₄²⁻, F⁻, Cl⁻, Br⁻, I⁻, BF₄⁻, AsF₆⁻, SbF₆⁻, CH₃CO₂⁻, CF₃SO₃⁻, (CF₃SO₂)₂N⁻, (CF₃SO₂)₃C⁻, and CF₃COO⁻.

Embodiment 16 provides the cured composition of any one of embodiments 9-15, wherein A⁻ is Br⁻.

Embodiment 17 provides the cured composition of any one of embodiments 9-16, wherein the compound of formula I is present in an amount of 0.1 to 1.25% (w/w) relative to the weight of the dental composition.

Embodiment 18 provides the cured composition of any one of embodiments 9-17, wherein the compound of formula I is present in an amount of 0.2 to 1% (w/w) relative to the weight of the dental composition.

Embodiment 19 provides the cured composition of any one of embodiments 9-18, wherein the compound of formula I is selected from the group consisting of.

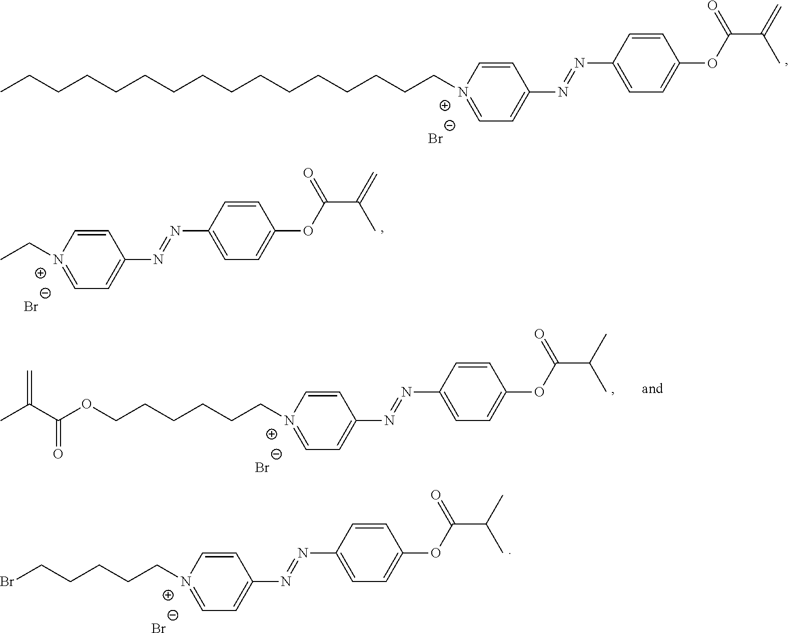

Embodiment 20 provides a method of using a dental composition, the method comprising:
applying a dental composition to an oral surface to form a coated oral surface, wherein the dental composition comprises a dental adhesive or resin and at least one compound of formula I,

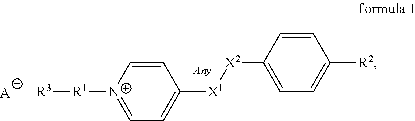

wherein:
   A⁻ is an anion;
   X¹ and X² are each independently selected from the group consisting of O, N, C, CH, and CH₂;
   [symbol] represents a single, double, or triple bond;
   R¹ is selected from the group consisting of —CₙH₍₂ₙ₊₁₎ and —CₙH₍₂ₙ₋₁₎;
   R² is selected from the group consisting of —OC₂₋₈ alkenyl, methacrylate, acrylate, styrene, vinyl benzyl, isobutyrate, and —OH;
   n is 2,3,4,5, 6, 7, 8, 9, 10, 11, 12, 13, 14, 15, 16, 17, 18, 19, or 20;
   wherein the composition optionally comprises at least one third moiety R³ attached to R¹, wherein R³ is selected from the group consisting of methacrylate, acrylate, styrene, vinyl benzyl, F, Cl, Br, and I;
   wherein the compound of formula I is present in an amount of 0.1 to 1.25% (w/w) relative to the weight of the dental composition;
and curing the coated oral surface.

Embodiment 21 provides the method of embodiment 20, wherein the oral surface is a tooth surface.

Embodiment 22 provides the method of any one of embodiments 20-21, wherein the oral surface is a dental article selected from the group consisting of crowns, bridges, veneers, inlays, onlays, fillings, and orthodontic appliances.

Embodiment 23 provides the method of any one of embodiments 20-22, wherein the orthodontic appliance is selected from the group consisting of orthodontic brackets, buccal tubes, lingual retainers, orthodontic bands, bite openers, buttons, and cleats.

Embodiment 24 provides the method of any one of embodiments 20-23, wherein [symbol] represents a double bond.

Embodiment 25 provides the method of any one of embodiments 20-24, wherein X¹ is N and X² is N.

Embodiment 26 provides the method of any one of embodiments 20-25, wherein A⁻ is selected from the group consisting of NO₃⁻, NO₂⁻, SCN⁻, CN⁻, PO₄³⁻, SO₄²⁻, F⁻, Cl⁻, Br⁻, I⁻, BF₄⁻, AsF₆⁻, SbF₆⁻, CH₃CO₂⁻, CF₃SO₃⁻, (CF₃SO₂)₂N⁻, (CF₃SO₂)₃C⁻, and CF₃COO⁻.

Embodiment 27 provides the method of any one of embodiments 20-26, wherein A- is Br—.

Embodiment 28 provides the method of any one of embodiments 20-27, wherein the compound of formula I is present in an amount of 0.1 to 1.25% (w/w) relative to the weight of the dental composition.

Embodiment 29 provides the method of any one of embodiments 20-28, wherein the compound of formula I is present in an amount of 0.2 to 1% (w/w) relative to the weight of the dental composition.

Embodiment 30 provides the method of any one of embodiments 20-29, wherein the compound of formula I is selected from the group consisting of:

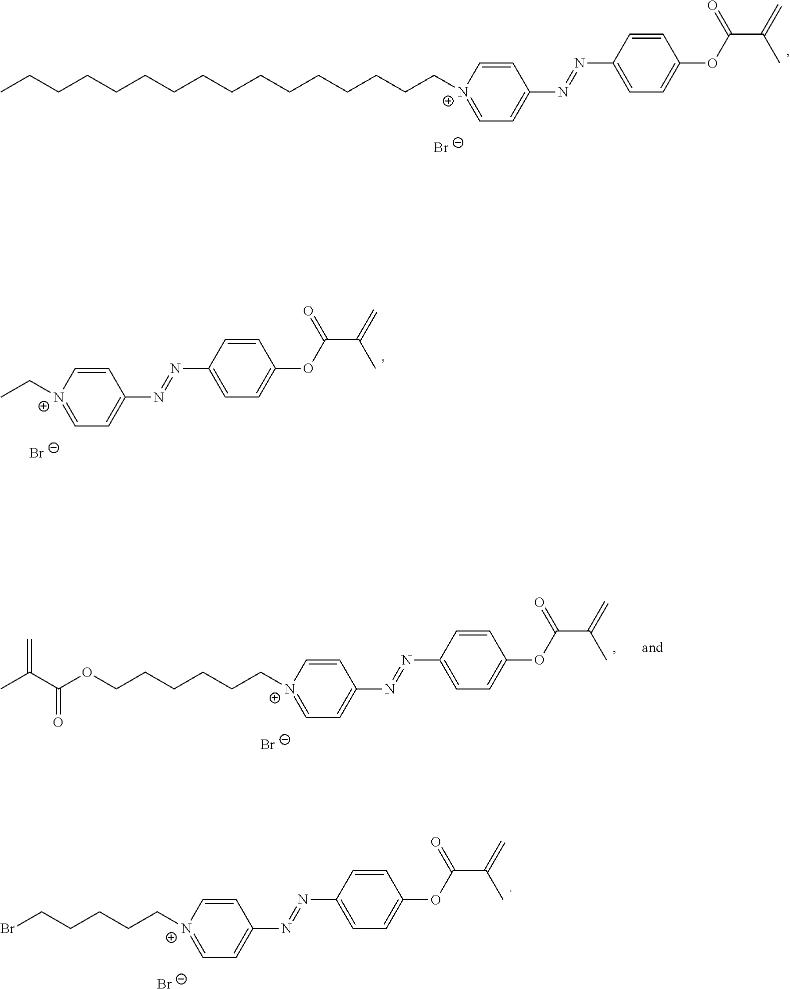

What is claimed is:

1. A dental composition comprising:
a dental adhesive or resin, and
at least one compound of formula I, 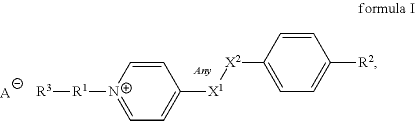
wherein:
A⁻ is an anion;
X¹ and X² are each independently selected from the group consisting of O, N, C, CH, and CH₂; [symbol]
represents a single, double, or triple bond;
R¹ is selected from the group consisting of —CₙH₍₂ₙ₊₁₎ and —CₙH₍₂ₙ₋₁₎;
R² is selected from the group consisting of —OC₂₋₈ alkenyl, methacrylate, acrylate, styrene, vinyl benzyl, isobutyrate, and —OH;
n is 2, 3, 4, 5, 6, 7, 8, 9, 10, 11, 12, 13, 14, 15, 16, 17, 18, 19, or 20;
wherein the composition optionally comprises at least R³ attached to R¹,
wherein R³ is selected from the group consisting of methacrylate, acrylate, styrene, vinyl benzyl, F, Cl, Br, and I; and
wherein the compound of formula I is present in an amount of 0.1 to 1.25% (w/w) relative to the weight of the dental composition.

2. The dental composition of claim 1, wherein [symbol] represents a double bond.

3. The dental composition of claim 1, wherein X¹ is N and X² is N.

4. The dental composition of claim 1, wherein A⁻ is selected from the group consisting of NO₃⁻, NO₂⁻, SCN⁻, CN⁻, PO₄³⁻, SO₄²⁻, F⁻, Cl⁻, Br⁻, I⁻, BF₄⁻, AsF₆⁻, SbF₆⁻, CH₃CO₂⁻, CF₃SO₃⁻, (CF₃SO₂)₂N⁻, (CF₃SO₂)₃C⁻, and CF₃COO⁻.

5. The dental composition of claim 4, wherein A⁻ is Br⁻.

6. The dental composition of claim 1, wherein the compound of formula I is present in an amount of 0.1 to 1.25% (w/w) relative to the weight of the dental composition.

7. The dental composition of claim 1, wherein the compound of formula I is present in an amount of 0.2 to 1% (w/w) relative to the weight of the dental composition.

8. The dental composition of claim 1, wherein the compound of formula I is selected from the group consisting of:

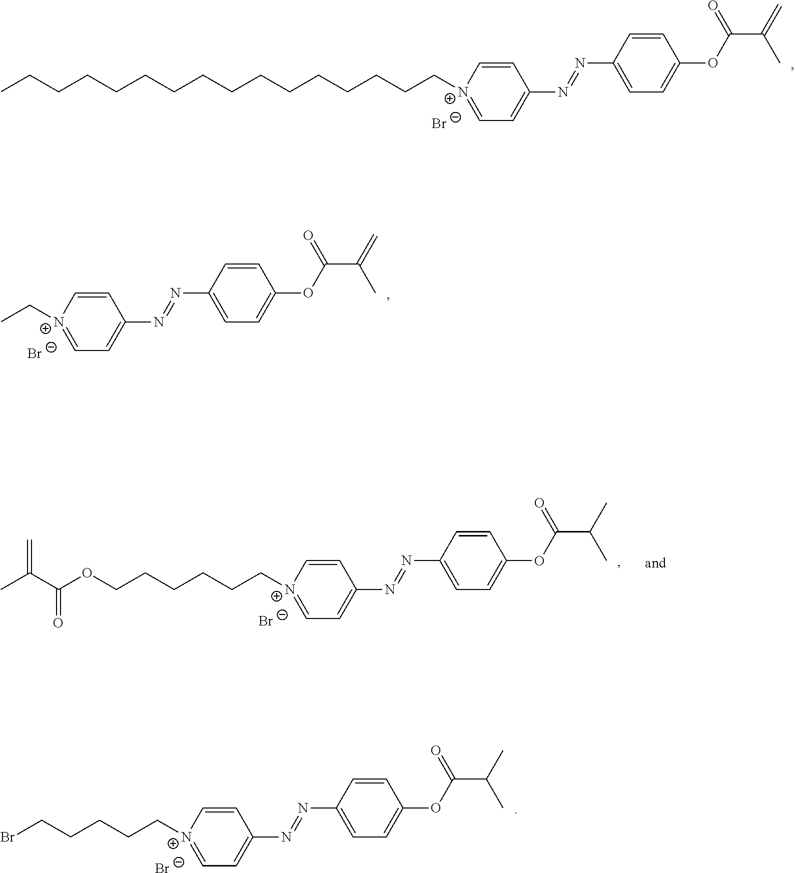

9. A cured composition comprising a polymerized product of a dental adhesive or resin and at least one compound of formula I, 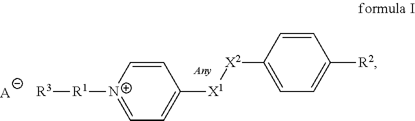
wherein:
A⁻ is an anion;
X¹ and X² are each independently selected from the group consisting of O, N, C, CH, and CH₂; [symbol]
represents a single, double, or triple bond;
R¹ is selected from the group consisting of —CₙH₍₂ₙ₊₁₎ and —CₙH₍₂ₙ₋₁₎;
R² is selected from the group consisting of —OC₂₋₈ alkenyl, methacrylate, acrylate, styrene, vinyl benzyl, isobutyrate, and —OH;
n is 2,3,4,5, 6, 7, 8, 9, 10, 11, 12, 13, 14, 15, 16, 17, 18, 19, or 20;
wherein the composition optionally comprises at least one R³ attached to R¹, wherein R³ is selected from the group consisting of methacrylate, acrylate, styrene, vinyl benzyl, F, Cl, Br, and I; and
wherein the compound of formula I is present in an amount of 0.1 to 1.25% (w/w) relative to the weight of the dental composition.

10. The cured composition of claim 9, wherein the polymerized product has a higher degree of vinyl conversion as compared to an equivalent cured composition lacking the compound of formula I.

11. The cured composition of claim 9, wherein the polymerized product has a higher biostability as compared to an equivalent cured composition lacking the compound of formula I.

12. The cured composition of claim 9, wherein polymerized product has a higher durability as compared to an equivalent cured composition lacking the compound of formula I.

13. The cured composition of claim 9, wherein [symbol] represents a double bond.

14. The cured composition of claim 9, wherein X¹ is N and X² is N.

15. The cured composition of claim 9, wherein A⁻ is selected from the group consisting of NO₃⁻, NO₂⁻, SCN⁻, CN⁻, PO₄³⁻, SO₄²⁻, F⁻, Cl⁻, Br⁻, I⁻, BF₄⁻, AsF₆⁻, SbF₆⁻, CH₃CO₂⁻, CF₃SO₃⁻, (CF₃SO₂)₂N⁻, (CF₃SO₂)₃C⁻, and CF₃COO⁻.

16. The cured composition of claim 15, wherein A⁻ is Br⁻.

17. The cured composition of claim 9, wherein the compound of formula I is present in an amount of 0.1 to 1.25% (w/w) relative to the weight of the dental composition.

18. The cured composition of claim 9, wherein the compound of formula I is present in an amount of 0.2 to 1% (w/w) relative to the weight of the dental composition.

19. The cured composition of claim 9, wherein the compound of formula I is selected from the group consisting of:

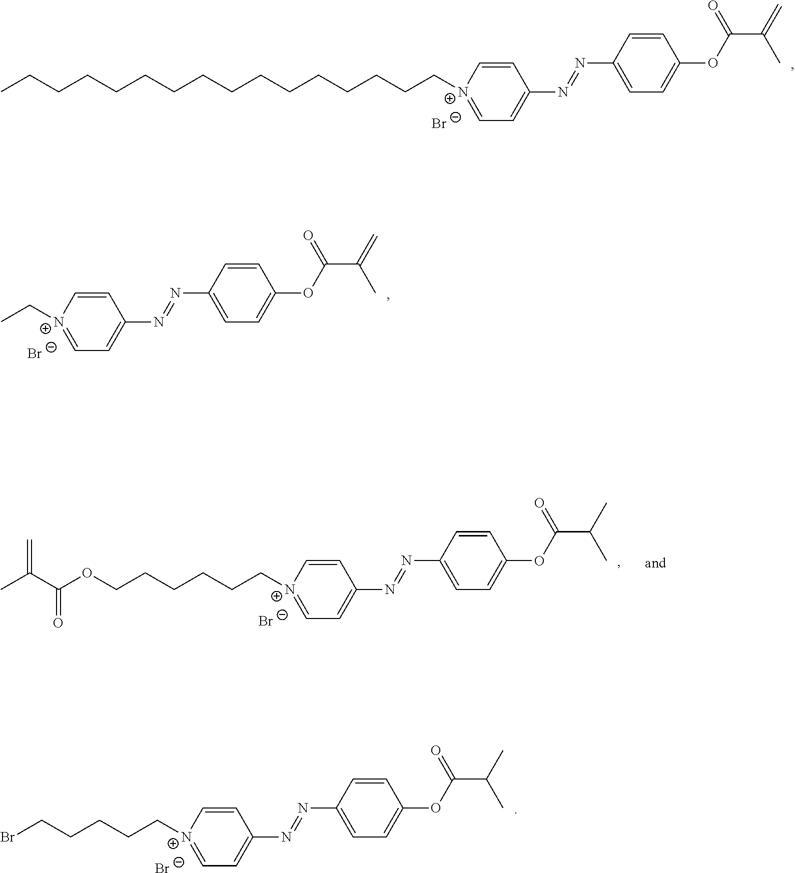

20. A method of using a dental composition, the method comprising:
applying a dental composition to an oral surface to form a coated oral surface, wherein the dental composition comprises a dental adhesive or resin and at least one compound of formula I, 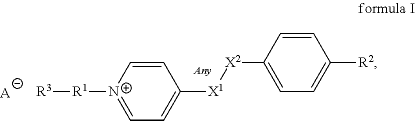
wherein:
A⁻ is an anion;
X¹ and X² are each independently selected from the group consisting of O, N, C, CH, and CH₂; [symbol]
represents a single, double, or triple bond;
R¹ is selected from the group consisting of —CₙH₍₂ₙ₊₁₎ and —CₙH₍₂ₙ₋₁₎;
R² is selected from the group consisting of —OC₂₋₈ alkenyl, methacrylate, acrylate, styrene, vinyl benzyl, isobutyrate, and —OH;
n is 2, 3, 4, 5, 6, 7, 8, 9, 10, 11, 12, 13, 14, 15, 16, 17, 18, 19, or 20;
wherein the composition optionally comprises at least one R³ attached to R¹, wherein R³ is selected from the group consisting of methacrylate, acrylate, styrene, vinyl benzyl, F, Cl, Br, and I;
wherein the compound of formula I is present in an amount of 0.1 to 1.25% (w/w) relative to the weight of the dental composition;
and
curing the coated oral surface.

21. The method of claim 20, wherein the oral surface is a tooth surface.

22. The method of claim 20, wherein the oral surface is a dental article selected from the group consisting of crowns, bridges, veneers, inlays, onlays, fillings, and orthodontic appliances.

23. The method of claim 22, wherein the orthodontic appliance is selected from the group consisting of orthodontic brackets, buccal tubes, lingual retainers, orthodontic bands, bite openers, buttons, and cleats.

24. The method of claim 20, wherein [symbol] represents a double bond.

25. The method of claim 20, wherein X¹ is N and X² is N.

26. The method of claim 20, wherein A⁻ is selected from the group consisting of NO₃⁻, NO₂⁻, SCN⁻, CN⁻, PO₄³⁻, SO₄²⁻, F⁻, Cl⁻, Br⁻, I⁻, BF₄⁻, AsF₆⁻, SbF₆⁻, CH₃CO₂⁻, CF₃SO₃⁻, (CF₃SO₂)₂N⁻, (CF₃SO₂)₃C⁻, and CF₃COO⁻.

27. The method of claim 26, wherein A⁻ is Br⁻.

28. The method of claim 20, wherein the compound of formula I is present in an amount of 0.1 to 1.25% (w/w) relative to the weight of the dental composition.

29. The method of claim 20, wherein the compound of formula I is present in an amount of 0.2 to 1% (w/w) relative to the weight of the dental composition.

30. The method of claim 20, wherein the compound of formula I is selected from the group consisting of: [chemical structure]
